(19) United States
(12) Patent Application Publication
(10) Pub. No.: US 2025/0050344 A1
(43) Pub. Date: Feb. 13, 2025

(54) SUBSTRATE TRAYS AND METHODS OF USE

(71) Applicant: BASF Agricultural Solutions Seed US LLC, Florham Park, NJ (US)

(72) Inventor: Punika PHUWANTRAKUL, Gent (BE)

(21) Appl. No.: 18/723,249

(22) PCT Filed: Dec. 20, 2022
(86) PCT No.: PCT/EP2022/086997

§ 371 Date: Jun. 21, 2024

(30) Foreign Application Priority Data
Dec. 21, 2021 (EP) 21216423.0

(51) Int. Cl. B01L 3/00

(52) U.S. Cl. CPC B01L 3/5085; B01L 2200/0689; B01L 2300/042; B01L 2300/0829; B01L 2300/0851

(57) ABSTRACT

Described herein are trays for storing, developing, analyzing, and transferring biological materials, such as biological samples. The trays comprise a plurality of compartments and can include a substrate contained within one or more compartments. Each compartment comprises a top open end, a body portion sidewall, and a base wall having at least one aperture. The base wall has an exterior portion and a removable seal can be disposed upon the exterior portion of the base wall. Systems including the trays and methods for developing, analyzing, and transferring biological materials using the trays are also provided herein.

### FIELD OF THE INVENTION

Described herein are substrate trays and methods for developing, analyzing, and transferring biological materials using the substrate trays.

### BACKGROUND

For numerous applications in biotechnology, biological material may be stored, preserved, developed, grown, incubated, transferred, or transported in or on a substrate. Often such substrate is held in multi-compartment trays, e.g., for handling, identification, analysis of samples, or other storage or processing needs.

Research, development, transport, and storage of biological material samples continue to require complementary labware, such as sample trays, that can be useful in high-throughput or automated processes, to allow for simple, quick, and accurate handling, processing, analysis, transport, and the like, of samples of biological material. There exist many different sample tray requirements for the plethora of different cell and tissues types, different experimental processes, different shipping conditions, and different analytical requirements. Existing sample trays may not be optimized for high throughput or automated analytical, growth, transport, or similar processes.

### SUMMARY

Thus, there remains a need for versatile substrate trays that satisfy a variety of conditions suitable for various intended applications in incubation, culturing, propagation or micropropagation, storing, transporting, and analyzing biological materials. There also remains a need for trays that can be easily used in various stages of biological material growth and analysis processes, including in automated and high-throughput processes. For example, it may be desirable to have trays that allow for automated sample analyses, automated picking or removal from tray compartments, or transferring or transplanting of biological materials. In particular, there remains a need for trays having compartments that can provide independent, fillable compartments with capabilities for segregating sample compartments while also providing multi-sides access to such compartments. Additionally, there remains a need for trays that can provide easy access to compartment contents for removal or analysis of the substrate or biological material contained within the compartments. These and other objectives are solved by the present invention as herein further described.

In one aspect, provided herein is a tray comprising:
a plurality of compartments, each compartment comprising
a top open end;
a body portion sidewall; and
a base wall having at least one aperture, wherein the base wall has an exterior portion, and
a removable fluid tight or selective fluid tight seal disposed on the exterior portion of the base wall of at least one compartment.

In some embodiments, the tray can optionally include one or more of the following features. The aperture can be a single aperture, and, optionally, the aperture can be disposed in the center of the base wall. A ratio of a surface area of the base wall measured in the plane of the base wall to a surface area of the aperture measured in the plane of the base wall can be from about 1.2:1 to about 5:1. The seal can be flexible. The seal can comprise a peelable seal. The seal can have a pull tab. The seal can be optically clear or transparent or opaque. The seal can be removably affixed to the exterior portion of the base wall by an adhesive, a heat seal, or a cold seal. The body portion sidewall can slope inward from the top open end to the base wall. The body portion sidewall can slope inward from the top open end to the base wall with a slope of from about 0.5 degrees to about 5 degrees. At least a portion of the tray can be adapted to engage with a tray lid. The tray can be a plant tray having a top side and a bottom side. The top side of the tray can comprise a lip portion adapted to engage with a tray lid. The tray can be constructed of a material selected from polypropylene, polyethylene, polyvinylchloride, polystyrene, polymethylmethacrylate, polycarbonate, polytetrafluoroethylene, polydimethylsiloxane, polysulfone, or combinations thereof. In some embodiments, the lid can be constructed of or comprise transparent or semi-transparent material. In some embodiments, the lid can be constructed of or comprise an opaque material. The tray and the compartments can be constructed as a single piece of material. The tray can further comprise a tray frame, and each compartment can be independently constructed of a separate piece of material from the tray frame. Each independent compartment can be removable. The tray can have from 2 to 384 compartments. The seal can be an independent seal independently disposed on each compartment, such that a tray having a plurality of compartments can have a plurality of seals, each independently disposed on each compartment. The seal can be a single seal disposed across 2 or more of the compartments. The seal can be a single seal disposed across the plurality of compartments. The tray can further comprise a substrate independently disposed within each compartment. The substrate can be selected from a bacterial culture media, a yeast culture media, a plant culture media, and an animal culture media. Optionally the substrate can comprise one or more gelling agents selected from agar, carrageenan, gellan gum, alginic acid and its salts, agarose, and combinations thereof. The substrate can be a liquid, gel, semi-solid or solid at room temperature.

In another aspect, provided herein is a system comprising:
at least one automated platform selected from an automated substrate dispenser, an automated biological material picker or gripper, an automated analytical platform, an automated sensor, and an automated transplanter; and
a tray according to the description herein.

In another aspect, provided herein is a method for producing a tray, comprising:
applying a removable seal to an exterior portion of a base wall of at least one compartment of a tray comprising:
   a plurality of compartments, each compartment comprising
   a top open end;
   a body portion sidewall; and
   a base wall having at least one aperture; and
sealing the seal to the exterior portion of the base wall of the at least one compartment such that a fluid tight seal is formed over the at least one aperture.

In some embodiments, the method can optionally include one or more of the following features. The method can further comprise filling at least one compartment with substrate. The substrate can be selected from a bacterial culture media, a yeast culture media, a plant culture media, and an animal culture media, optionally wherein the substrate comprises one or more gelling agents selected from agar, carrageenan, gellan gum, alginic acid and its salts, agarose, and combinations thereof. The substrate can be a gel at room temperature. The filling of the at least one compartment can be performed by an automated filling process.

In another aspect, provided herein is a method comprising:
placing biological material in or on the substrate in at least one compartment of a tray as described herein, wherein the tray comprises a substrate independently disposed within each compartment; and
further comprising a step selected from: storing the biological material in the tray for a period of time, transporting the tray with the biological material from a first location to a second location, allowing the biological material to incubate for a period of time, analyzing at least a portion of the biological material, or combinations thereof.

In another aspect, provided herein is a method comprising:
filling at least one compartment of a tray as described herein with substrate;
placing biological material in or on the substrate in at least one compartment of the tray; and
further comprising a step selected from: storing the biological material in the tray for a period of time, transporting the tray with the biological material from a first location to a second location, allowing the biological material to incubate for a period of time, analyzing at least a portion of the biological material, or combinations thereof.

An automated substrate dispenser can optionally be used to fill the at least one compartment of the tray.

In some embodiments, these two methods can optionally include one or more of the following features. The methods can comprise the step of analyzing at least a portion of the biological material, and wherein analyzing comprises:
positioning at least one sensor at a location directly above the top open end of a target compartment of the tray, or positioning at least one sensor at a location directly below the aperture of a target compartment of the tray; and
obtaining information about the biological material or the substrate from the at least one sensor.

The methods can further comprise removing the seal from the target compartment. The methods can comprise the step of analyzing at least a portion of the biological material, and wherein analyzing comprises:
at least partially removing a sample comprising at least a portion of the biological material, with or without at least a portion of the substrate, from a target compartment of the tray;
positioning at least one sensor in a position that allows collection of information from the sample; and
obtaining information about the biological material or the substrate from the at least one sensor.

Obtaining information about the biological material or the substrate from the at least one sensor can comprise moving the sensor in a 360 degree rotation around at least one axis of the sample. Removing the sample can comprise gripping the biological material or the substrate or a combination thereof through the top open end of the target compartment and pulling at least a portion of the biological material, with or without at least a portion of the substrate, out of the target compartment through the top open end of the target compartment. The gripping can be performed by an automated gripper. Removing the sample can comprise:
removing the seal from a target compartment of the tray to yield an open-aperture target compartment; and
inserting a pushing structure into the open-aperture target compartment through the aperture, thereby pushing at least a portion of the substrate and the biological material together through the top open end of the open-aperture target compartment.

Removing the seal can comprise peeling or pulling the seal off of the exterior portion of the base wall of the compartment. Removing the seal can comprise cutting at least a portion of the seal.

The cutting at least a portion of the seal can be performed with the pushing structure while inserting the pushing structure. The pushing structure can have a pushing surface that is approximately the same size and shape as the surface area of the aperture, or from about 0.05% to about 10% smaller than the surface area of the aperture. The pushing structure can be an automated pushing structure. The sensor can be an automated sensor or a sensor having automated movement capability. The sensor can be selected from a camera, a thermometer, a multispectral camera, or combinations thereof. The information can include information about the physiochemical status of the biological material. The information can include information selected from water content, chemical composition or partial chemical composition, phenotype, genotype, temperature, color, or size of at least a portion of the biological material. The method can further comprise transferring at least a portion of the biological material, with or without at least a portion of the substrate, to a location outside of the tray. The location outside the tray can be selected from a second tray, a receptacle, an analysis platform, a petri dish, or a plate comprising wells. The biological material can be a seed, a plant, or a plant part, and wherein transferring comprises transplanting the seed, plant, or plant part into a plant pot, a plug tray, or a plant tray. The transplanting can comprise using an automated transplanter. The placing can comprise using an automated placing mechanism. The biological material can be selected from an eukaryotic cell, a plant cell, an animal cell, a mammalian cell, a prokaryotic cell, a primary cell, a tumor cell, a stem cell, a genetically altered cell, a transformed cell, and an immortalized cell, a tissue or portion of a tissue selected from plant tissue or animal tissue, a plant seed, a protoplast, a cytoplast, a plant organ, or a plant part. The biological material can be selected from a seed or a plant part selected from a cutting, an embryo, a regenerated embryo, a colony, a microspore, a callus, or an anther.

The trays, including trays with or without lids, and methods described herein can have several benefits. First, the removable seal or seals positioned on the exterior portion of the base wall of one or more compartments of the tray provides a simple way to physically access the biological material sample and associated substrate through the bottom of the compartment. This can be useful for a number of applications. For example, access to the bottom of the compartment can provide capability for accessing and extracting a sample for analysis from deeper within the substrate rather than on top of the substrate, such as where root samples may be desired in the case of plant growth applications, or where tissue or cells growing within a substrate is more desirable for sampling than tissue or cells growing on top of a substrate. As another example, physical access to the bottom of the compartment can increase ease of removal of the entire sample (e.g., biological material and puck of associated substrate held within the compartment) for relocation of the sample to a different tray or storage apparatus, or to a final end-use location such as transplantation, or for emptying and re-use of the trays for further samples, or for removal of the sample for three-dimensional analysis, such as imaging or other sensor-based probing of the sample. The trays described herein aid removal and physical access to the bottom of the sample by way of a peelable seal on the bottom of the tray (disposed on an exterior portion of the base wall of at least one compartment). Removal of the seal reveals an aperture in the base wall of each compartment, thereby releasing any capillary or vacuum-like effect of a compartment having the seal in a sealing configuration, and providing access through the aperture into the bottom of the compartment. A portion of the sample can then be removed through the aperture, or the sample can be pushed up by pushing through the aperture onto the bottom of the sample such that the sample is then pushed at least partially through the top open end of the compartment, allowing the sample to then be fully removed from the compartment (and subsequently analyzed or transferred) or allowing the sample to be analyzed (such as, e.g., imaged) while at least a portion of the sample is sticking up through the top open end of the compartment.

Second, the removable seal or seals positioned on the exterior portion of the base wall of one or more compartments of the tray provide a simple way to access the sample for analysis, by, e.g., creating sensor access points or openings through which light can pass. For example, the relatively large aperture exposed upon removal of the seal can provide access for light necessary for, e.g., viewing, imaging, or other sensing of the sample. This can be particularly useful in cases where a light blocking material is needed during culture of the sample. In such cases, the tray and seal and an optional lid can all be created from light blocking materials, but upon removal of the seal and optional lid, the samples could remain within the tray and be imaged because light can pass through both the aperture and the top open end of the compartment. Removal of the seal and subsequent revealing of the aperture also allows an opening through which the bottom of the sample can be viewed and analyzed (e.g., imaging, temperature measurements, color analyses, etc.) while the sample remains within the compartment of the tray.

Third, the removable seal can provide support and a fluid tight (or selective fluid tight) seal for the base of the compartment in order to contain a substrate, including, e.g., liquid media, as it is dispensed into the compartments of the tray. Thus the removable seal can keep the substrate supported during liquid-phase filling and subsequent hardening of the substrate (in cases where liquid, gel, semi-solid, or solid substrate is desired), and keep the substrate segregated such that each compartment independently contains substrate that is not in fluid communication with another compartment's substrate, while also providing open access to the bottom of the substrate, semi-solid substrate, or hardened substrate after removal of the seal. In some embodiments, this segregation of the compartments can aid in experimental design, allowing different samples, treatments, substrates, conditions, and combinations thereof for each isolated compartment in a single tray, while also providing easy access to each compartment from multiple directions (after removal of the removable seal).

Fourth, because the compartments can be isolated from one another during growth, experimentation, transport, storage, and the like, as well as provide multi-directional access to each compartment, the trays described herein can be useful in tracking and tracing of samples within complex experimental designs. Further, the trays described herein therefore can also provide easy selection of target samples from within the experimental layout, as well as easy analysis of samples and identification of target samples after analysis. For example, if further growth, experimentation, or transplantation or a target sample is desired, the target samples or samples can be easily selected from among the tray compartments and, because of the isolation of each compartment, be advanced for further steps in a desired process.

Fifth, in some embodiments, each compartment can act as a plant tissue culture vessel, providing isolated compartments for micropropagation, plant tissue culture, and the like, which allows for isolated experimentation in each compartment, precise and simplified analysis of the cultured matter, such as roots plant embryos, protoplasts, and the like, through both the top open end and the aperture in the base wall, simplified selection and removal or manipulation of target samples, and combinations thereof.

Sixth, because the compartments can be isolated from one another and from an environment exterior to the compartments, e.g., through the presence of the removable seal, the trays described herein can be used in processes requiring sterile conditions (for example, the trays can be used in embodiments with a lid, which, in combination with the removable seal can aid in maintaining sterile conditions by, e.g., preventing microbial contamination).

Seventh, because the trays comprise seals over the bottom apertures, the trays described herein can be useful as independent trays (and not solely as an insert), without the need for an additional tray or box to set the trays in to prevent, e.g., spill or leakage of substrate through the apertures during filling, storage, growing, etc.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

For the terms “for example” and “such as,” and grammatical equivalences thereof, the phrase “and without limitation” is understood to follow unless explicitly stated otherwise. As used herein, the term “about” is meant to account for variations due to experimental error. As used herein, the singular forms “a,” “an,” and “the” are used interchangeably and include plural referents unless the context clearly dictates otherwise.

The details of one or more implementations of the subject matter of this disclosure are set forth in the accompanying drawings and the description. Other features, aspects, and advantages of the subject matter will become apparent from the description, the drawings, and the claims.

### BRIEF DESCRIPTION OF THE FIGURES

FIG. 1A is a top perspective view of one illustrative embodiment of a tray as described herein.

FIG. 1B is a top plan view of the tray of FIG. 1A.

FIG. 1C is a bottom perspective view of the tray of FIGS. 1A-1B in an exploded configuration.

FIG. 1D is a bottom plan view of the tray of FIG. 1A-1B.

FIG. 2A is a cross-sectional perspective view of a portion of one illustrative embodiment of a tray as described herein.

FIG. 2B is a cross-sectional schematic view of a portion of one illustrative embodiment of a tray as described herein.

FIG. 3 is a top perspective view of one illustrative embodiment of a tray as described herein.

FIG. 4: is a cross-sectional perspective view of a portion of one illustrative embodiment of a tray with an engaged lid as described herein

FIG. 5A and FIG. 5B show examples of filters 133 on top of the lid in different sizes in a perspective view of the tray with an engaged lid as described herein.

FIG. 6: is a cross-sectional perspective view of a portion of one illustrative embodiment of a tray with an engaged lid with a filter 133 as described herein.

FIG. 7 and FIG. 8 are a top plan views of the tray with compartments in described sizes, shapes and numbers as described herein.

FIG. 9 is a perspective view of the tray with an engaged lid with a filter 133 as described herein.

FIG. 10 is a perspective view of the tray with an opened lid with a filter 133 as described herein.

FIG. 11: is a cross-sectional perspective view of a portion of one illustrative embodiment of a tray with an engaged lid with a filter 133 as described herein.

FIG. 12 is a perspective view of the tray with an engaged lid with a filter 133 and elements 134, e.g., for keeping a distance to the bottom of a tray stabled on top of the lid.

### DETAILED DESCRIPTION

In the following description of illustrative embodiments, reference is made to the accompanying figures of the drawing which form a part hereof, and in which are shown, by way of illustration, specific embodiments. It is to be understood that other embodiments may be utilized, and structural changes may be made without departing from the scope of the present invention.

The trays described herein may be useful in a variety of applications in which a biological material is to be placed in or on a substrate, such as a liquid or gel substrate. One example of such uses is cell culture. Another example of such uses is tissue culture. In some cases, biological material may be placed on or in a substrate in order to perform analytic procedures, such as imaging. In some cases, trays described herein can be useful in combinations of multiple processes, such as storage, identification, growth or culture, analysis, and transport of biological material including, e.g., an eukaryotic cell, a plant cell, an animal cell, a mammalian cell, a prokaryotic cell, a primary cell, a tumor cell, a stem cell, a genetically altered cell, a transformed cell, and an immortalized cell, or a tissue or portion of a tissue selected from plant tissue or animal tissue, a plant seed, a protoplast, a cytoplast, a plant organ, or a plant part. In some embodiments, the biological material is selected from a seed or a plant part selected from a cutting, an embryo, a regenerated embryo, a colony, a microspore, a callus, or an anther, or combinations thereof.

Additionally, the trays described herein can be useful in automated processes, such as where biological growth or analysis processes utilize an automated system. One example of such an automated process can include imaging of biological material within its sample substrate. For example, the trays described herein provide a variety of sample imaging or sensing capabilities, including imaging a biological sample from a camera or sensor positioned above a top open end of a compartment of the tray, imaging a biological sample from a camera or sensor positioned below an aperture in a base wall of a compartment of the tray, or removal of the substrate and biological material sample (e.g., as a gel puck or disc or other three-dimensional shape) from a compartment, either by pulling the substrate and sample through the top open end of the compartment, or by pushing the substrate and biological material sample up through the top open end of the compartment by pushing on the substrate through an aperture in the base wall of the compartment.

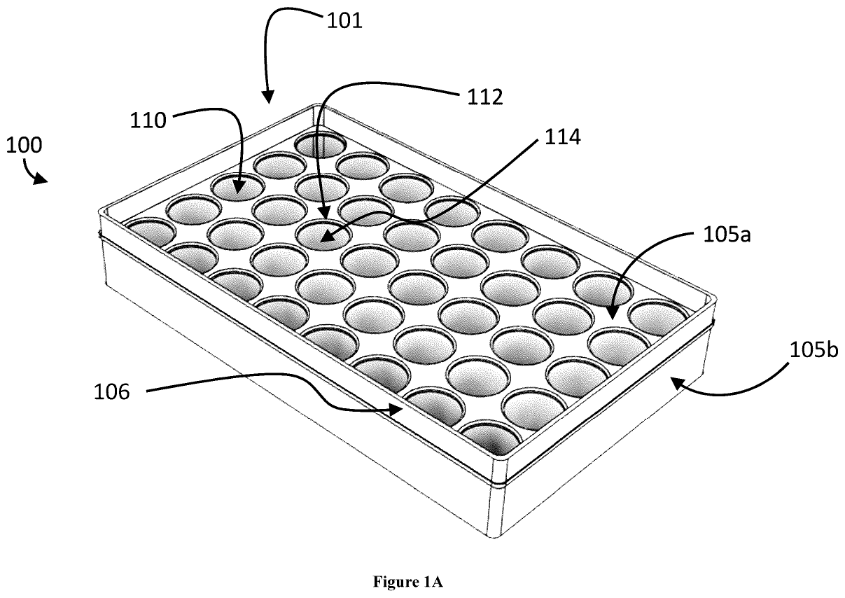

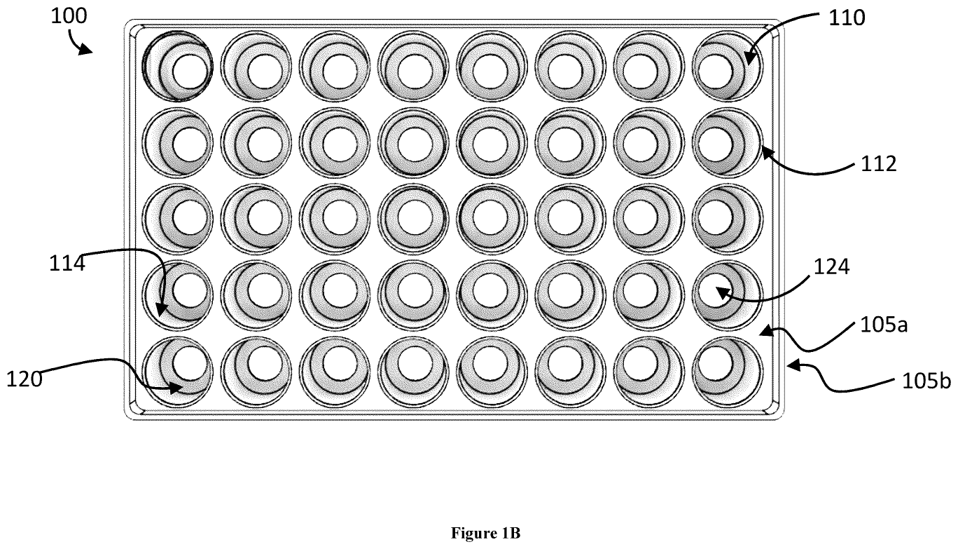

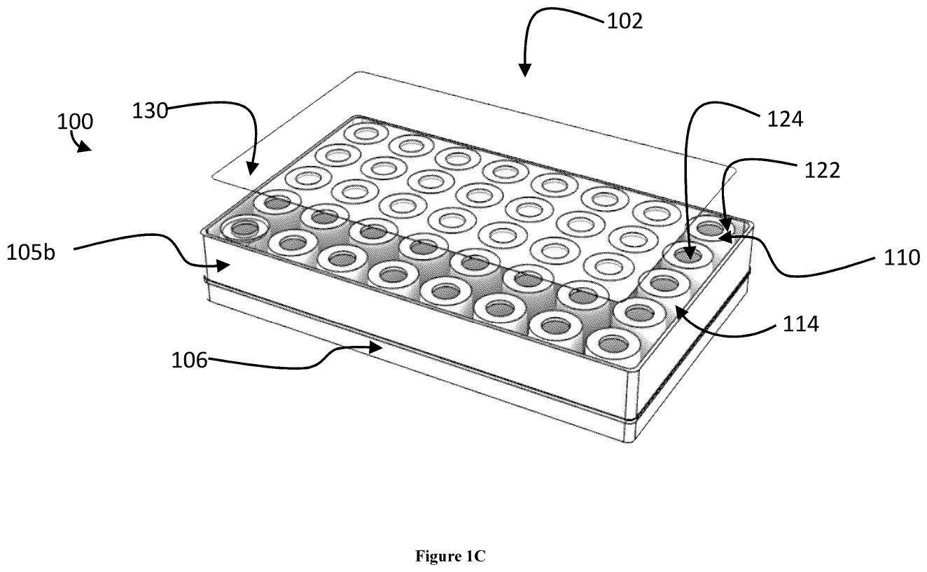

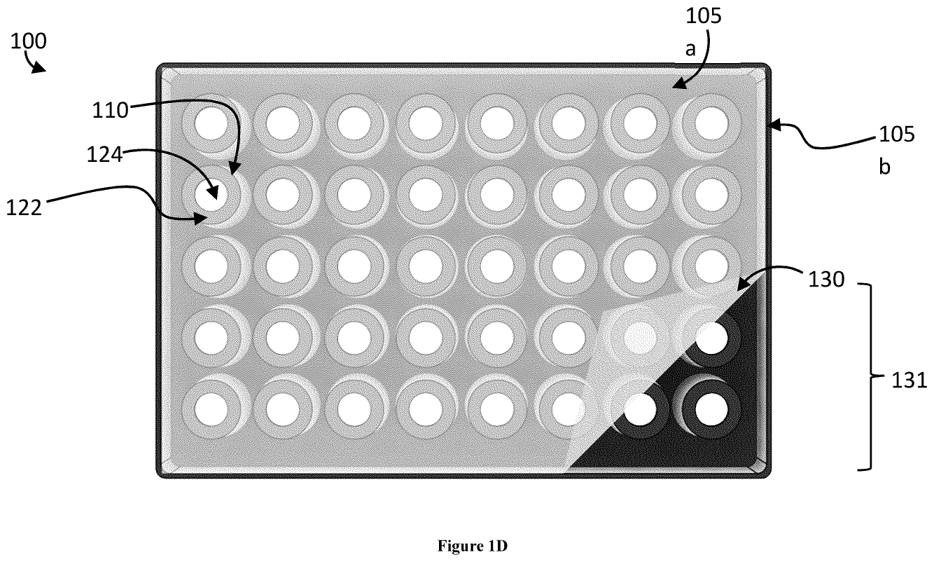

One illustrative embodiment of a tray as described herein is depicted in connection with FIGS. 1A-1D. FIG. 1A depicts a top perspective view of tray 100 showing top side 101 of tray 100, FIG. 1B depicts a top plan view of tray 100, FIG. 1C depicts a bottom perspective view of tray 100 in an exploded configuration showing bottom side 102 of tray 100, and FIG. 1D depicts a bottom plan view of tray 100. The tray 100 comprises a plurality of compartments 110, each compartment comprising a top open end 112, a body portion sidewall 114, and a base wall 120 having at least one aperture 124. The base wall 120 has an exterior portion 122. The tray further comprises at least one removable fluid tight or selective fluid tight seal 130 disposed on the exterior portion 122 of the base wall 120 of at least one compartment 110. Each tray 100 can have any suitable number of compartments 110. In some embodiments, a tray 100 can have from two (2) compartments 110 to three hundred and eighty-four (384) compartments 110. In some embodiments, a tray 100 can have 2, 3, 4, 5, 6, 7, 8, 9, 11, 15, 16, 24, 25, 30, 32, 35, 36, 40, 48, 96, 104, 105, or 384 compartments 110. Tray 100 can be of any suitable shape. The shape of tray 100 can be selected based on the desired use, shape and dimensions of existing machinery used in desired processes, dimensions of storage and shipping facilities, etc. In some embodiments, tray 100 can have a rectangular shape, a square shape, or an elliptical shape.

Seal 130 is depicted in an exploded configuration in FIG. 1C, and in FIG. 1D, seal 130 is shown in a partially sealing position, with a corner portion 131 in an upturned configuration as some embodiments might look during peeling away of seal 130 from the bottom 102 of the tray 100 or from exterior portion 122 of base wall 120 of the compartments 110. Seal 130 can, in some embodiments, be a single piece of that covers the aperture 124 of multiple or of all compartments 110. In some embodiments, seal 130 can comprise multiple individual seals (not shown), each seal covering aperture 124 of one compartment 110 or a limited number of total compartments 110. In some embodiments, seal 130 can be an independent seal independently disposed on each compartment 110, such that a tray 100 having a plurality of compartments 110 has a plurality of seals (not shown), each independently disposed on each compartment 110. In some embodiments, seal 130 can be a single seal disposed across two or more of the compartments 110. In some embodiments, seal 130 can be a single seal disposed across the plurality of compartments 110. Seal 130 can, in some embodiments, comprise a pull tab (not shown) or other structure that allows easy gripping and removal of seal 130.

The tray 100 includes a frame comprising a top surface 105a, and optionally, a perimeter wall 105b. In some embodiments, the tray is a single continuous unit such that the top surface 105a, the compartments 110, and the option perimeter wall 105b are all constructed out of the same continuous piece of material. Alternatively, one or more parts of the tray 100, such as, e.g., compartments 110, can each be independently constructed of separate pieces of material. For example, in some embodiments, the optional perimeter wall 105b of the frame can be constructed of a separate piece of material, and can optionally be removable or detachable from the top surface 105a of the frame. As another example, in some embodiments, each compartment 110 can be individually constructed of a separate piece of material. In some embodiments, compartments 110 individually constructed of a separate piece of material can be removable or detachable from the top surface 105a of the frame of tray 100. The material of each individual piece that makes up the tray can be the same or can be composed of different materials from one another.

In some embodiments, at least a portion of tray 100 can optionally be adapted to engage with a tray lid. For example, in some embodiments, tray 100 can comprise a lip 106, or other suitable structure, adapted to engage with a lid. In some embodiments, tray 100 can include a lid (not shown in FIG. 1A-1D). The lid 190 and tray 100 can be connected so that the lid is reversible but firmly connected to the tray. In one embodiment, the lid 190 and tray 100 are connected by a suitable device for holding lid and tray, e.g., a clip or a fastener, or by other systems that allows a firm but reversible engaging between lid and tray, e.g., by a click or snap mechanism. In some embodiments, the tray 100 can comprise a locking mechanism (not shown), that can prevent the lid (not shown) from disengaging with the tray, for example during transport or movement of the tray. In some embodiments, the optional locking mechanism can provide an airtight seal. An airtight seal can, in some embodiments, allow the trays described herein to maintain sterile conditions by, e.g., preventing microbial contamination (for example, from fungi, bacteria, viruses, or mycoplasma). In some embodiments, the optional locking mechanism prevents or reduces the likelihood of the lid being opened or released during a drop. Suitable locking mechanisms for lid and tray connections are known to the skilled person and such suitable mechanisms and any adaptations will be apparent based on the description of the trays and lids herein. In some embodiments, the tray 100 can comprise a removable or fixed connection to the lid (not shown in FIG. 1A-1D). For example, tray and lid are connected to each other by a hinge or fitting that allows a limited angle of opening of the lid to the tray. The tray 100 and lid 190 can be connected by a hinge that allows movement about one axis of rotation. In another embodiment, the hinge is a multi-joint hinge comprising multiple axes or joints. For example, a suitable hinge, such as but not limited to a multi-joint hinge, allows the opening of the lid to swing outward without contacting the tray. In one embodiment, the tray and the lid are connected by a hinge comprising a damper. Advantageously, the opening of the lid can be done with one hand. In one embodiment, the hinge is combined with a spring or compression element that allows the lid to be opened one-handedly by the tensile forces that occur, for example with a compression hinge. In one embodiment, the lid swings open after being the connection between lid and tray is unlocked on lid or tray. The hinge can determine the opening angle of the lid. For example, the opening angle may be only a few degrees. Opening the lid by more than 45°, for example 60°, 70°, 80°, 90°, 1000 or 1200 or more allows access to the contents of the box when the lid is open but still connected to the tray. The maximal opening between tray and lid is depending on the weight distribution between the lid and tray. The opening angle can be selected such that the tray-lid-combination does not tip over when the lid is open or opened. An opening more than 1200 is thus possible. For example, a multi-joint hinge can be used to open the lid so that the lid or parts of the lid rest on the same surface as the tray. The hinge can also be selected to press the closed lid against the tray, e.g., by means of a spring or another pressure element. Pressing the lid against the tray allows to seal the lid tighter as without pressure. When the lid is opened, the opening can occur against the pressure of the hinge, e.g., up to an advantageous angle, and then the hinge latches in a defined angle. For example, the lid can be pushed or pressed out of the latched position, the lid then can automatically close again, for example in a damped manner if a damped hinge is used.

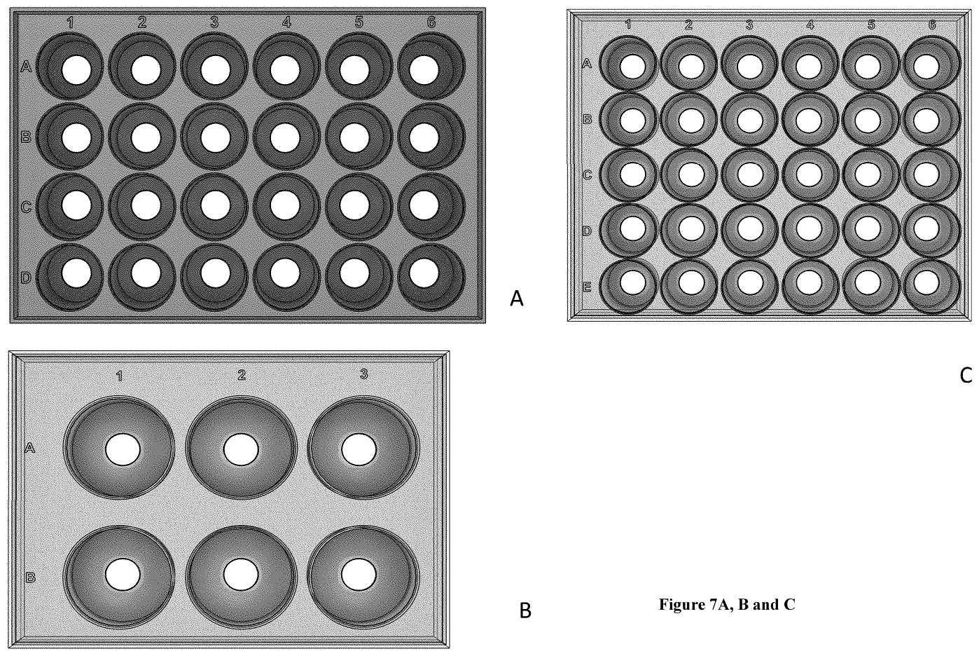

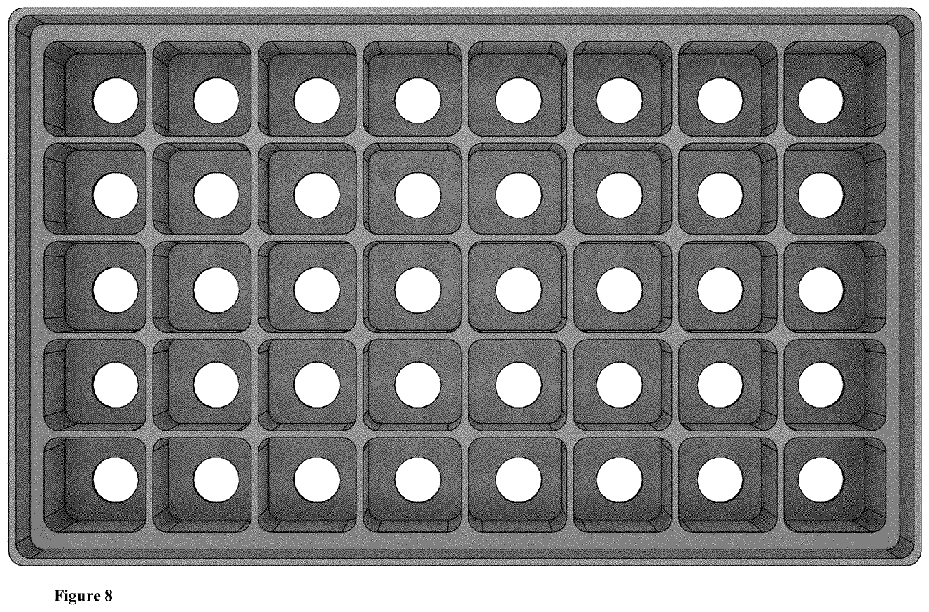

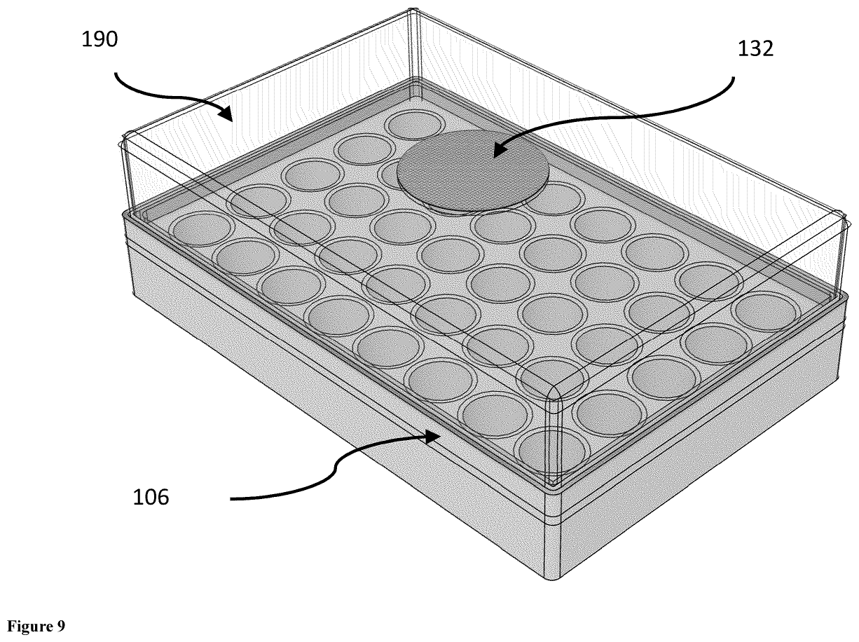

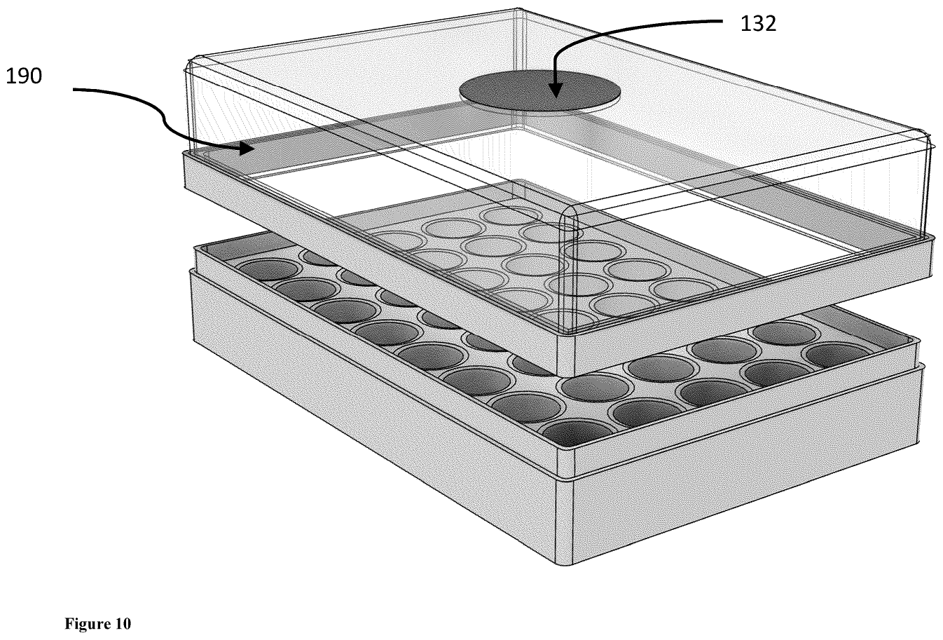

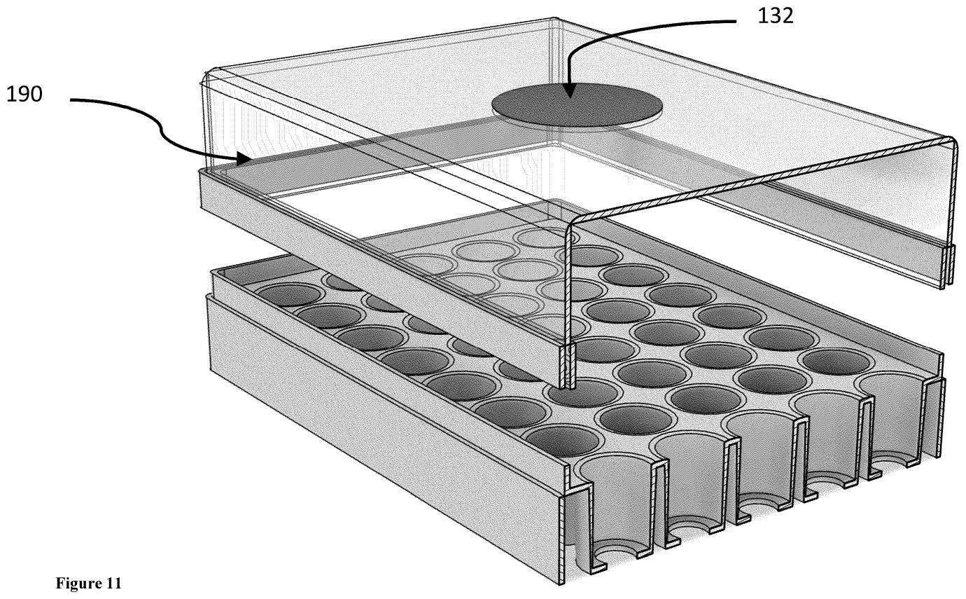

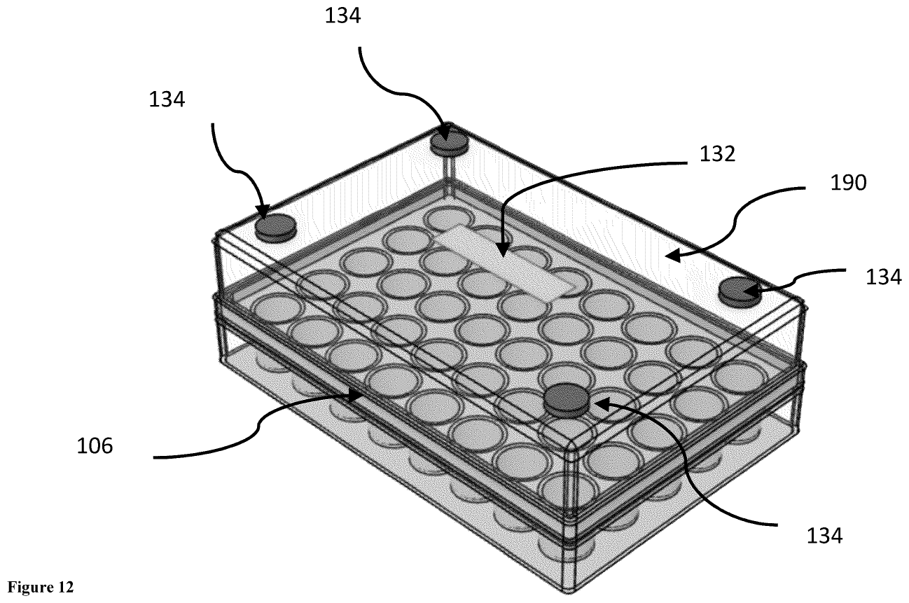

Although the embodiment depicted in FIGS. 1A-1D shows a top open end 112 of compartment 110 that is generally elliptical in shape (e.g., circular), and the depicted embodiment of compartment 110 is generally cylindrical in shape, including body portion sidewall 114 that is generally cylindrical in shape and base wall 120 that is generally elliptical (e.g., circular) in shape, other compartment 110 shapes can be used in the trays described herein, for examples, the upper and/or the lower end of compartment 110 can have the shape of a circle, square, rectangle, oval, pentagon, exagon, octagon, etc. Accordingly, the compartment 110 can have the shape of or similar to a cube, cylinder, prism, e.g. a rectangular prism, cone, pyramid, or combinations thereof. For instance, aperture 124 can have a quadrilateral shape while compartment 110 has an elliptical shape. FIGS. 7 and 8 illustrate different shapes of compartment 110. For example, in some embodiments, body portion sidewall 114 can be comprised of multiple walls or multiple wall pieces that come together to form a compartment shape, and base wall 120 can, in some embodiments, be quadrilateral in shape (e.g. square, rectangular, etc.). For example, in some embodiments, body portion sidewall 114 can comprise four sidewalls or four sidewall portions (of the same continuous sidewall) that form, together with, e.g., a square or rectangular base wall 120, a generally cubic or rectangular cubic volume within compartment 110. Essentially, the compartments 110 of the trays described herein may take any suitable shape that includes at least one body portion sidewall that, together with the base wall and, in some embodiments the seal, defines an internal volume of the compartment 110 in which a substrate (e.g., liquid, gel, soil, and the like) can be contained. Compartment 110 can, in some embodiments, further contain biological material disposed on or within the substrate, such that compartment 110 contains a sample comprising substrate and biological material disposed on or within the substrate.

While the illustrative tray 100 embodiment depicted in FIGS. 1B-1D shows an aperture 124 in base wall 120 that is generally elliptical in shape (e.g., circular), aperture 124 may take any suitable shape. for examples, the aperture 124 can have the shape of a circle, square, rectangle, oval, pentagon, exagon, octagon, etc. For example, aperture 124 can, in some embodiments, be quadrilateral in shape (e.g., square, rectangular, etc.). While the depicted embodiment shows aperture 124 having a generally comparable shape to top open end 112, body portion sidewall 114, and base wall 120, aperture 124 need not have a shape comparable to base wall 120, body portion sidewall 114, or top open end 112 of compartment 110. For example, in some embodiments, aperture 124 can, in some embodiments, have a shape that is different than base wall 120, body portion sidewall 114, or top open end 112 of compartment 110. In some embodiments, for instance, aperture 124 can have a quadrilateral shape while base wall 120 has an elliptical shape, or vice versa.

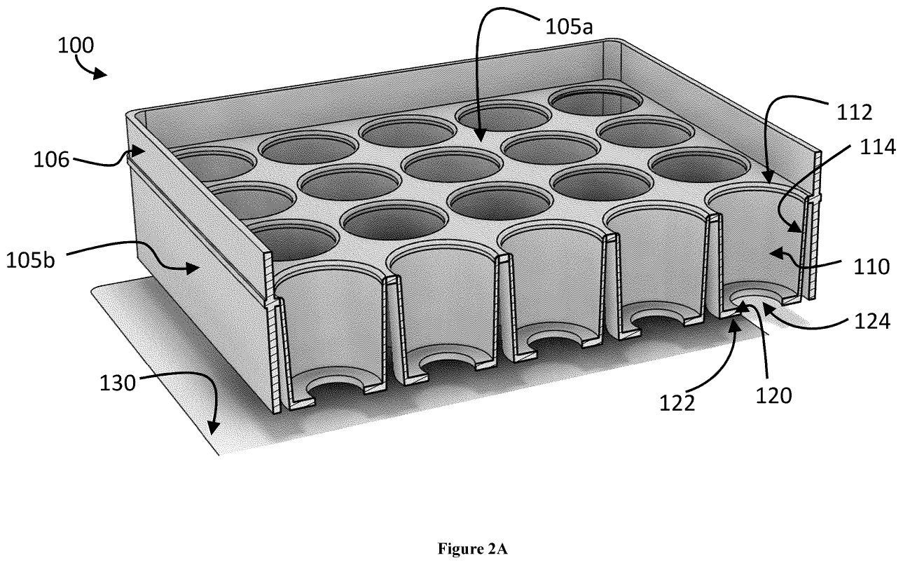

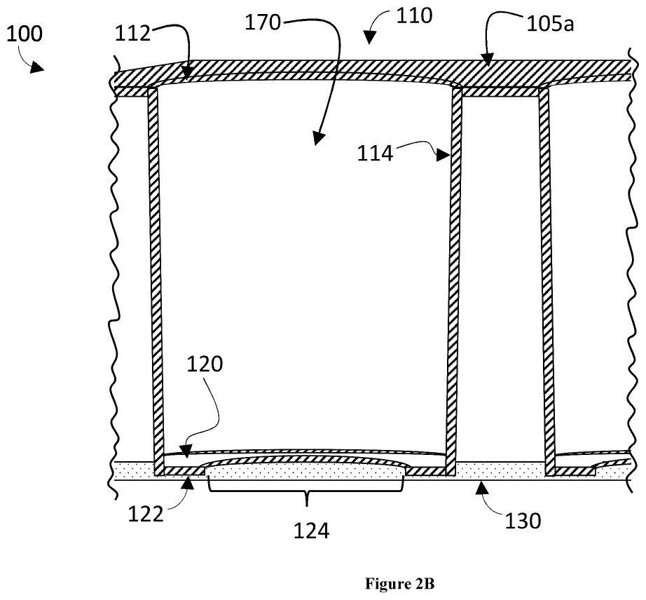

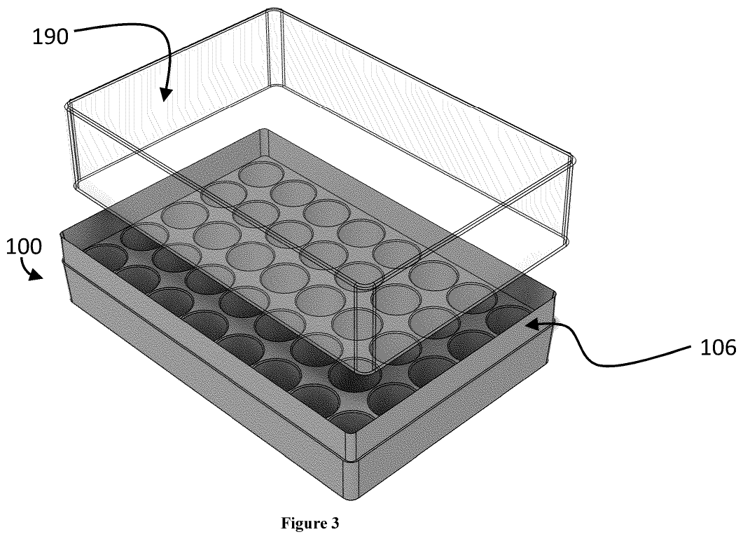

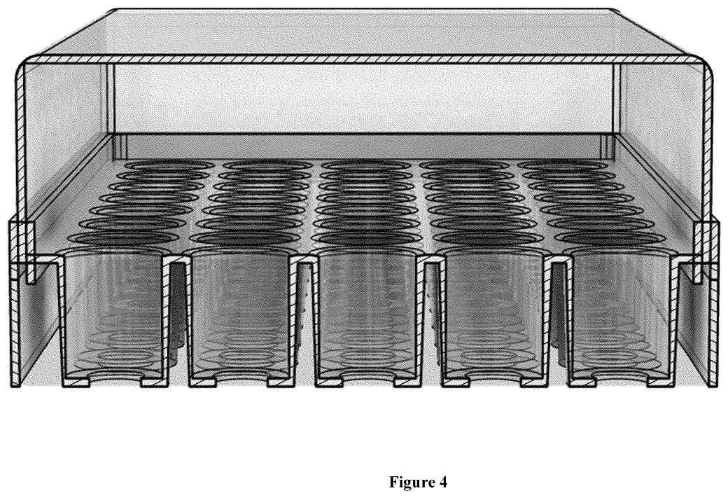

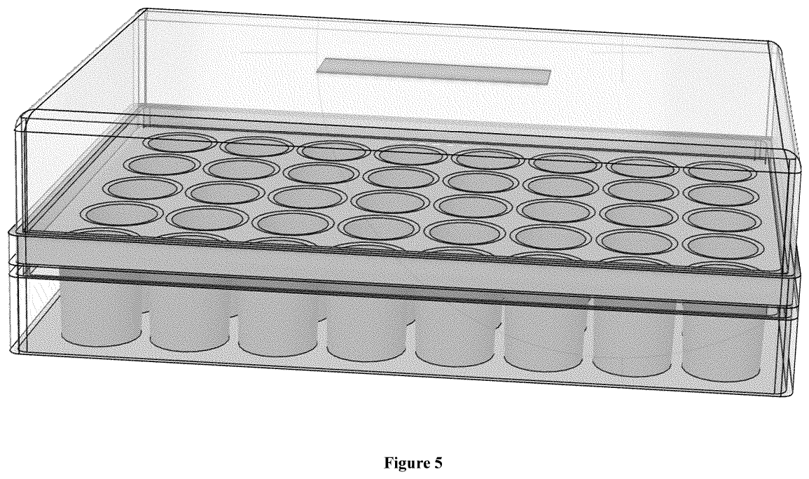

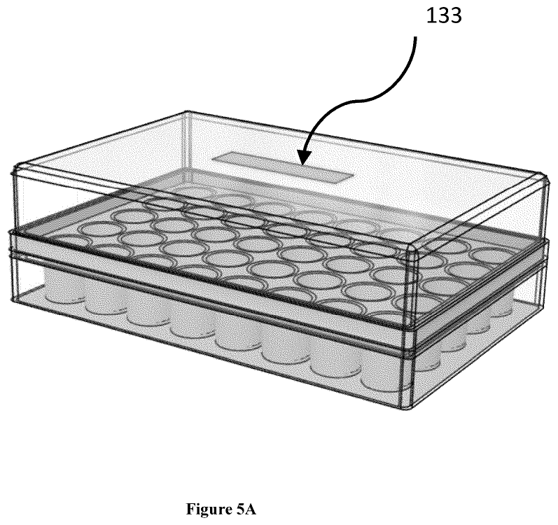

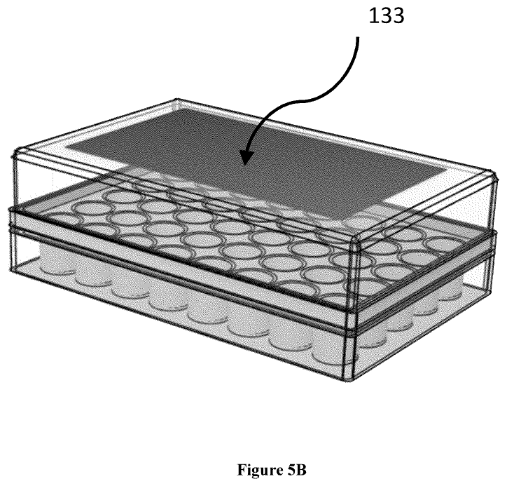

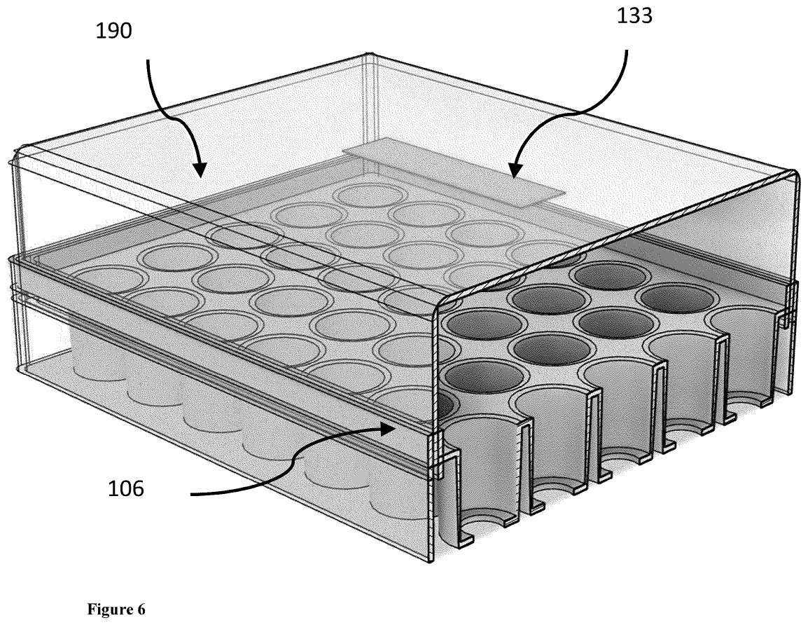

FIG. 2A depicts a cross-sectional perspective view of a portion of the tray 100 in an exploded configuration, with tray 100 comprising a frame comprising a top surface 105a, and optionally, a perimeter wall 105b. Tray 100 optionally includes a lip 106, or other suitable structure, adapted to engage with a lid or other structure. Tray 100 comprises a plurality of compartments 110, each compartment having a top open end 112, a body portion sidewall 114, a base wall 120 having an exterior portion 122, an aperture 124 in base wall 120. Seal 130, shown here in an exploded configuration, is a removable fluid tight or selective fluid tight seal. When in a sealing configuration, seal 130 is disposed on the exterior portion 122 of the base wall 120 of at least one compartment 110.

FIG. 2B is an enlarged cross-sectional schematic view of a portion of tray 100, comprising top surface 105a and compartment 110 having a top open end 112, a body portion sidewall 114, and a base wall 120, which together define an inner volume 170. Base wall 120 has an exterior portion 122 and an aperture 124 in base wall 120. Seal 130 is in a sealing position, covering aperture 124 and providing a fluid tight seal such that fluids cannot flow out of inner volume 170 of compartment 110 through aperture 124, or a selective fluid tight seal such that specific fluids, such as liquids, cannot flow out of inner volume 170 of compartment 110 through aperture 124 but other specific fluids, such as gases can.

Body portion sidewall 114 can, in some embodiments, slopes inward from the top open end 112 to the base wall 120, such that the inner volume 170 has a larger diameter (or dimension for non-elliptical configurations) at the top open end 112 than at the base wall 120. In some embodiments, the body portion sidewall 114 can slope inward from the top open end 112 to the base wall 120 with a slope of from about 0.5 degrees to about 5 degrees, from about 0.5 degrees to about 4 degrees, from about 0.5 degrees to about 3 degrees, from about 0.5 degrees to about 2.5 degrees, from about 0.5 degrees to about 2 degrees, from about 0.5 degrees to about 1.5 degrees, from about 0.5 degrees to about 1 degree, from about 1 degree to about 5 degrees, from about 1 degree to about 4 degrees, from about 1 degree to about 3 degrees, from about 1 degree to about 2.5 degrees, from about 1 degree to about 2 degrees, from about 1 degree to about 1.5 degrees.

In some embodiments, body portion sidewall 114 can be constructed such that it is smooth or flat, while in some embodiments, body portion sidewall 114 can be textured. For example, in some embodiments, body portion sidewall 114 can be corrugated such that, e.g., a textured pattern of raised vertical lines running from the top open end 112 to the base wall 120 are present. Other textured surfaces can be used for body portion sidewall 114 as desired.

Without wishing to be bound by theory, it is believed that in the sealing configurations described herein, the trays described herein provide some resistance to the expulsion of liquid, gel, semi-solid, or solid substrate contained within the inner volume 170 of compartment 110 when the tray is tipped from an upright position to a slanted or even upside down position, for example, during an accidental position shift during transport. Thus, the trays can, in some embodiments, maintain the gel, semi-solid, or solid substrate within the inner volume 170 of compartment 110 when tray 100 is in a non-upright position (e.g., a position at least 2, 3, 4, 5, 6, 7, 8, 9, 10, 15, 20, 25, 30, 35, 40, 45, 50, 55, 60, 65, 70, 75, 80, 85, 90, 100, 110, 120, 130, 140, 150, 160, 170, or 180 degrees from upright, or upside down) for at least some period of time, such as at least 10 seconds, at least 20 second, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, or at least 30 minutes. In some embodiments, the period of time for which the gel, semi-solid, or solid substrate is maintained within inner volume 170 of compartment 110 when tray 100 is in a non-upright position can be increased by or decreased by adding specific textures to the body portion sidewall. For example, corrugation or other texture can be added to the body portion sidewall. Without wishing to be bound by theory, it is believed that the increase in surface area created by use of a texture on the body portion sidewall can increase the friction between the body portion sidewall and the substrate, thus making it more difficult for the substrate to slide out of the compartment, for example, in the event of an accidental drop or tipping over of the tray. It is further believed that vertical correlation running from the base wall to the top open end can both aid in increasing surface area and increasing friction to prevent accidental dislodging of the substrate while also not preventing the purposeful pushing or removal of the substrate and/or sample out of the compartment for processes such as analysis, transplantation, and the like.

In some embodiments, aperture 124 can be a single aperture. In some embodiments, aperture 124 can be disposed in the center of the base wall. In some embodiments, aperture 124 can be off-center with respect to the base wall. The shape and location of the aperture can be chosen based on the desired use. For example, in instances where automation machinery has a pushing structure with a square-shaped surface for pushing, e.g., a sample comprising a substrate and biological material disposed on or within the substrate out of compartment 110, the aperture 124 may be square in shape. The aperture 124 in base wall 120 of compartment 110 of the trays described herein has provides multiple advantages as described herein. For example, the aperture 124 should be large enough to allow access for sensors, or for pushing structures to push the sample out of the compartment 110, or to provide other benefits described herein. In addition, the base wall 120 should be sufficiently sized to prevent solid, semi-solid, or gel substrate (e.g., such as a substrate plug or puck) from falling out of the compartment 110 through the aperture 124 when the seal 130 is not present, to provide adequate fluid tight or selective fluid tight sealing when the seal 130 is disposed on and sealed to the exterior portion 122 of base wall 120, as well as provide adequate surface area for seal 130 so that seal 130 does not dislodge or unseal from the exterior portion 122 of base wall 120 during filling of the compartment 110. In some embodiments, the coupling of the seal 130 to the exterior portion 122 of base wall 120 surrounding aperture 124 prevents or reduces the likelihood of migration of substrate out of compartment 110 (including migration of substrate from one compartment to another), and provides stable sealing even when pressure is exerted against seal 130 during filling of the compartment 110 with substrate. Thus the relative size of the aperture 124 and the relative size of the base wall 120 can have an important role in the function of the trays described herein. In some embodiments, the base wall 120 and aperture 124 can be sized such that a ratio of a surface area of the base wall 120 (excluding the aperture) measured in the plane of the base wall to a surface area of the aperture 124 measured in the plane of the base wall is from about 1:1 to about 5:1, from about 1.2:1 to about 5:1, from about 1.2:1 to about 1.5:1, from about 1.2:1 to about 2:1, from about 1.2:1 to about 2.2:1, from about 1.2:1 to about 2.5:1, from about 1.2:1 to about 3:1, from about 1.2:1 to about 3.5:1, from about 1.2:1 to about 4:1, from about 1.2:1 to about 4.5:1, from about 1.5:1 to about 4:1, from about 1.5:1 to about 3.5:1, from about 1.5:1 to about 3:1, from about 1.5:1 to about 2.5:1, from about 1.5:1 to about 2:1, from about 2:1 to about 5:1, from about 2:1 to about 4.5:1, from about 2:1 to about 4:1, from about 2:1 to about 3.5:1, from about 2:1 to about 3:1, or from about 2:1 to about 2.5:1. In some embodiments, the base wall 120 and aperture 124 can be sized such that a ratio of a surface area of the base wall 120 measured in the plane of the base wall to a surface area of the aperture 124 measured in the plane of the base wall is about 2:1. In some embodiments, the base wall 120 can extend inward toward the middle of the compartment 110 in the plane of the base wall about 13-16 mm from the body portion sidewall 114. In some embodiments, the aperture 124 can have a diameter of from about 6 to about 8 mm. In some embodiments, the aperture has a surface area at least 20% less than the surface area of the sum of the base wall and the aperture.

In general, the trays described herein can be useful for, e.g., storing, incubating, growing, transporting, or analyzing biological material such as cells and tissues. The compartments 110 of tray 100 can be filled with a substrate, e.g., growth media or storage substrate, and biological material can be placed on or within the substrate in the compartments. For example, the trays described herein can be useful for storing, incubating, growing, transporting, or analyzing biological material such as, but not limited to a cell selected from an eukaryotic cell, a plant cell, an animal cell, a mammalian cell, a prokaryotic cell, a primary cell, a tumor cell, a stem cell, a genetically altered cell, a transformed cell, and an immortalized cell, or a tissue or portion of a tissue selected from plant tissue or animal tissue, or plant seeds or plant parts (e.g., a cutting, an embryo, a regenerated embryo, a colony, a microspore, a callus, or an anther). The trays described herein can be useful, in some embodiments, for cell culture, tissue culture (including, e.g., plant micropropagation such as cutting regeneration, somatic embryogenesis, embryogenesis and double haploid production, androgenesis, and the like), biological material storage or transport, analysis of biological material, and the like.

The compartments 110 can, in some embodiments, contain substrate (e.g., at least partially filled or fully filled) such as a growth media. Non-limiting examples of substrate include growth media such as bacterial culture media, yeast culture media, plant cell culture media, and animal cell culture media. The substrate can be a liquid, gel, semi-solid, solid, loose solid, particulate, or the like. In some embodiments, the substrate can optionally comprise one or more gelling agents. Non-limiting exemplary gelling agents include agar, carrageenan, gellan gum, alginic acid and its salts, agarose, and combinations thereof. Other non-limiting exemplary substrates can include soil, vermiculite, clay, peat, foams, plant matter, animal matter, scaffolding, acellular scaffolding, polymeric material, and combinations thereof. In some embodiments, empty compartments 110 are at least partially filled, and in some embodiments are fully filled, with substrate after seal 130 has been disposed upon and sealed to the external portion 122 of base wall 120 to seal aperture 124 with a fluid tight or selective fluid tight seal. In some embodiments, when the seal 130 is in a sealing position, a liquid, semi-liquid, liquid-like, or flowable material can be poured into at least one compartment 110 to at least partially fill the compartment. In some embodiments, the liquid, semi-liquid, liquid-like, or flowable material can harden, partially harden, semi-harden, or become more gelled, gel-like, or solid-like after being poured into the at least one compartment 110. Alternatively, pre-formed solid or semi-solid substrate, including semi-solid gels, such as those in the form of a pre-made plug can, in some embodiments, be placed in the empty compartments 110, with or without the presence of seal 130. The substrate in the compartment 110 of the trays described herein is generally independently disposed within each compartment such that, when seal 130 is in a sealing position and thus sealing aperture 124 of one or more compartments 110, there is no physical or liquid communication between a sealed compartment and another compartment when the tray is in an upright and level position as shown in FIG. 1A. In some embodiments, gaseous communication may occur between the compartments, for example, due to the top open end 112 of each compartment or due to gas-permeable seals.

FIG. 3 depicts a top perspective view of tray 100 having an optional lid 190. Tray 100 can optionally have a lip 106 or other structure adapted to engage with lid 190. However, such engagement or structure is not necessary for embodiments of trays such as tray 100 that have a lid such as lid 190. For example, a lid may still be used for embodiments of trays that lack a specific structure for engaging with a lid. In some embodiments, a lid may simply rest on a portion of the tray. In some embodiments, a lid may be placed completely over a tray such tray 100 and rest on a surface external from the tray (i.e., not touch or engage with the tray at all). In one embodiment, a container comprising lid 190 and tray 100 can be set on top of another container comprising lid and tray 100. In one embodiment, the container comprising a tray with a lit is set on top, e.g. is stacked, such that the bottom of tray 100 of the upper container sits on top of the lid 190 of the lower container. For instance, two or more containers can be stabled, e.g., stacked. The containers that are stabled or stacked can but do not need to be identical in form or size. For instance, a tray 100 with an engaged lid 190 is set on top of the lid 190 of another tray 100 such that both containers are engaged.

The bottom or the top of containers comprising lid 190 and tray 100 can be designed such that a gap between the lid 190 of the lower unit and the bottom of the tray 100 of the upper unit allows a gas exchange. Such a gap may be provided, for example, by one or more spacers placed either between lid of the lower container and bottom of the upper container, or by one or more spacers build on or into the lid 190, or by one or more spacers that are fixed to or build into the bottom of tray 100, the one or more spacers allowing keeping a distance between the two containers. In one embodiment, the one or more spacers support the stacking of the upper container's tray bottom on top of the lid of lower container. For instance, one or more spacers of the lower container engage with, e.g., lock to, the upper unit in such a way, for example due to the shape of the spacers, that the stabled containers do not slip, e.g. the stack is stabilized but the containers are not fixed to each other.

The spacer can have any form, it is for example elliptical in shape (e.g., circular), and the depicted embodiment of compartment 134 is for instance cylindrical in shape, other spacers 134 can have the shape of a circle, square, rectangle, oval, pentagon, hexagon, octagon, etc. and can, for example, be located anywhere on the surface of the lid, allowing a stable sitting of the upper tray 100 on top of the lid 190 below.

The lid 190 and/or the tray 100 are made from a single material or combinations of materials. In one embodiment, the lid 190, or the tray, e.g., the lip 106, can have one or more opening 132 or 133, that allow gas exchange. In one embodiment, the openings are many very small or micro holes that allow gas exchange but no or little contamination. For example, the small or micro holes are located on the top of the lid 190 or at the side walls of the lid or in the lip 106. The holes can be designed such that a contamination of the samples in the compartments, e.g., with pathogens, is low. The opening or the openings can be sealed, e.g., with a filter. For example, the openings can be sealed with a seal as described for the aperture. In one embodiment, the opening or openings are sealed with a filter 133, that allows gas exchange.

A selective filter can prevent the flow or migration of fluids through or across the filter while allowing oxygen or other desired gases or humidity to pass through the filter into or out of the inner volume of the compartment. In one embodiment, the filter allows the selective exchange of gases like for example oxygen, CO₂ and/or humidity. Such selective filter can advantageously support incubation, growth, or analyses processes that require the influx of gases into the compartment or substrate. For example, in some embodiments of plant tissue culture, a gas permeable filter can allow proper levels of oxygen and carbon dioxide for cell or plant growth. In some embodiments, a selective filter can allow humidity to pass into or out of the compartment in a controlled manner, thus allowing humidity control for a given sample while also providing a fluid tight seal.

In some embodiments, the lip 106 or other suitable structure, may be adapted to engage with or support something other than a lid, such as a mechanism for holding a camera or other sensor, a mechanism or structure for supporting plant growth, a structure for further separating the space above compartments 110 from each other, and the like. In some optional embodiments, symbols, structures (e.g. raised or protruding structures, lines, dots, or the like), holes, or other markings (e.g., barcodes), or RFID structures can be present on the frame (not shown), such as on top surface 105a, perimeter wall 105b, lip 106, or compartments 110, or embedded in substrate within compartments 110. Embedded markers or identifiers can, in some embodiments, include a chip or RFID structure. In some embodiments, dyes or chemical markers can be added to the substrate in one or more compartments to act as an identifier, orientation marker, or the like, or to show a researcher which compartment contains or does not contain a target chemical marker or target process or genetic profile. The markings or other identifiers can, in some embodiments, assist with identification or tracking of the tray or of individual samples in each compartment, or with orientation of the tray, or combinations thereof. In some embodiments, analysis or operational machines can use the markers or identifiers to orient, select, analyze, move, etc. a sample or set of samples, or to orient a machine or portion of a machine. In some embodiments, the markings or structures can be used for alignment or other directional guiding of the trays in an automated system, for example, to ensure the trays are centered on a conveyor prior to interaction with a sensor or a gripper or a filler or other automated platform. As another example, the markings can provide an alignment specific to a sensor, or can be used in sensor data collection to provide spatial information, sample information, or the like. In some embodiments, the structures can also be used for physical engagement with, e.g., a sensor or an automated platform system, to ensure correct positioning or assist with other locational or orientation needs within the given system.

The trays described herein can be produced by any suitable means. In some embodiments, it may be preferred that all of the features depicted in FIGS. 1A-3, except for seal 130 or lid 190, be molded of the same material, e.g., a thermoplastic such as polypropylene, polyethylene, and combinations thereof. Such a construction is not, however, required and one or more of the different features may be constructed of different materials that are joined or connected together by any suitable technique or combination of techniques.

Described herein are methods for producing trays described herein, comprising applying a removable seal to an exterior portion of a base wall of at least one compartment of a tray comprising a plurality of compartments, each compartment comprising a top open end; a body portion sidewall; and a base wall having at least one aperture; and sealing the seal to the exterior portion of the base wall of the at least one compartment such that a fluid tight seal is formed over the at least one aperture. The method can, in some embodiments, further comprise filling at least one compartment with substrate. In some embodiments, the method comprises filling a plurality of or all of the compartments with substrate. The filling can, in some embodiments, be performed manually. Alternatively, in some embodiments, the filling of the at least one compartment is performed by an automated filling process. In some embodiments, the substrate is selected from bacterial culture substrate, yeast culture substrate, plant culture substrate, and animal culture substrate, optionally wherein the substrate comprises one or more gelling agents selected from agar, carrageenan, gellan gum, alginic acid and its salts, agarose, and combinations thereof. In some embodiments, the substrate can be a gel at room temperature. In some embodiments, the substrate is a flowable gel or liquid when poured into the compartments 110 of tray 100. In some embodiments, the substrate is a flowable gel or liquid when poured into the compartments 110 of tray 100 and subsequently hardens to a solid or a more solidified gel after being poured into the compartments of the tray. In some embodiments, seal 130 provides sufficient support and fluid tight or selective fluid tight sealing such that seal 130 does not break or disengage from its sealing position during filling of the compartments 110 with substrate. In some embodiments, the compartments can be partially filled with substrate or with a sample comprising substrate and a biological material (and, optionally, any desired reagents or additives). Alternatively, in some embodiments, the compartments 110 can be fully filled with substrate, or with a sample comprising substrate and a biological material (and, optionally, any desired reagents or additives).

Trays described herein can be constructed of any suitable material. In some embodiments, one or more portions of the tray is constructed of a material selected from polypropylene, polyethylene, polyvinylchloride, polystyrene, polymethylmethacrylate, polycarbonate, polytetrafluoroethylene, polydimethylsiloxane, polysulfone, or combinations thereof. In some embodiments, the tray can be constructed of a transparent or semi-transparent material. In some embodiments, the tray can be constructed of an opaque material. One advantage of the aperture in the base wall is that the trays can be constructed of opaque or color materials without affecting some visualization, light-dependent, or analysis processes because sufficient light can pass through the top open end and the aperture in the base wall to allow for visual observation, sensor-based observation or analyses, or other light-dependent processes. Thus, the breadth of materials usable in constructing the trays described herein can be greater than other trays used for similar light-dependent processes. This can also support usage of biodegradable, recyclable, or recycled materials for increased sustainability of the tray. The combination of the aperture and top open end of the compartments can also support processes that are light-sensitive or require darkness, such as certain growth stages of biological material, or when using light-degradable compounds in treatment of the biological materials or gels contained within the compartments. In such embodiments, the trays and, in some embodiments the seals, can be constructed of dark materials to support the desired light-sensitive processes, then the aperture in the base wall and the top open end can be used to provide sensor access to the materials or gel for analysis even in cases of analyses that require light or visualization. In some embodiments, the trays can be constructed of colored materials (with each tray having a single continuous color or multiple colors, such as, e.g., compartments having different colors) for color-coding or identification of trays, tray types, tray materials, samples, sample types, substrate types, or the like.

The removable seal 130 or seals can be constructed of any suitable material. Exemplary non-limiting materials include poly olefins such as polyethylene or polypropylene, polyesters such as polyethylene terephthalate, nylon, polystyrene, polysulfone, polytetrafluoroethylene, a silicone copolymer, and combinations thereof. In some embodiments, the seal can be a simple film, while in other embodiments the seal can comprise a multilayer film for added functionality, including preserving sterility during transport of trays having gas-permeable seals, light blocking properties, and the like. An example of such material is a multilayer film prepared by laminating a vapor-deposited layer of a metal such as aluminum or a layer including metal foil and a resin film. In some embodiments, the seal or seals can be constructed of an optically clear or transparent material. In some embodiments, the seal or seals can be constructed of an opaque or colored material. In some embodiments, colored seals may be used for color coding or identification or compartments, trays, materials, sample types, substrate types, or the like, or for analyses or growth or incubation processes in which a color filter is desired. In some embodiments, colored or opaque seals may be used to provide light-blocking capability during light sensitive processes.

In some embodiments, the seal or seals can be constructed of a flexible material such as a thin, flexible polymeric material. In some embodiments, the seal or seals can be constructed of a rigid material. In some embodiments, the seal can have a thickness of from about 0.02 mm to about 1 mm, from about 0.03 mm to about 1 mm, from about 0.04 mm to about 1 mm, from about 0.05 mm to about 1 mm, from about 0.075 mm to about 1 mm, from about 0.08 mm to about 1 mm, from about 0.1 mm to about 1 mm, from about 0.1 mm to about 0.9 mm, 0.1 mm to about 0.8 mm, from about 0.1 mm to about 0.7 mm, from about 0.1 mm to about 0.6 mm, 0.1 mm to about 0.5 mm, from about 0.1 mm to about 0.4 mm, from about 0.1 mm to about 0.4 mm, 0.1 mm to about 0.3 mm, from about 0.05 mm to about 0.5 mm, from about 0.05 mm to about 0.3 mm, from about 0.05 mm to about 0.2 mm.

The seal or seals can be removably affixed or bonded to the exterior portion of the base wall of one or more compartments by any suitable technique or combination of techniques that provides a fluid tight seal or selective fluid-tight seal over the aperture in the base wall of one or more compartments. Exemplary, non-limiting techniques include, e.g., heat sealing, adhesives, cold sealing, chemical welding, ultrasonic sealing, and the like. In some embodiments, the seal or seals can be removably affixed or bonded to or disposed upon the base wall of one or more compartments with an adhesive. In some embodiments, the seal or seals can be removably affixed or bonded to or disposed upon the base wall of one or more compartments with a heat seal. In some embodiments, the seal 130 is a peelable seal, e.g., a seal that can be peeled up or peeled off in order to release the seal. In some embodiments, the portion of seal 130 disposed across aperture 124 can be optionally coated with or constructed from a material that provides non-stick properties or easy release such that the seal is prevented from sticking to the substrate during removal or peel off of the seal. In some embodiments, seal 130 can be removably bonded to another portion of the tray in addition to exterior portion 122 of base wall 120 of compartment 110. For example, in some embodiments, seal 130 can be removably affixed or bonded to a portion of the tray frame, such as a portion of perimeter wall 105b.

As used herein, the term “fluid” refers to all forms of flowable materials including liquids, gases, dispersions, emulsions, and free-flowing solids or powders. For example, fluids can include flowable materials such as water, liquid culture media substrate, flowable gels, non-hardened gels, soil or soil-like substances. A fluid tight seal can prevent the flow or migration of all or most fluids through or across the seal indefinitely or for a specified period of time. A selective fluid tight seal can prevent the flow or migration of selected fluids through or across the seal indefinitely or for a specified period of time while also allowing the flow or migration of other fluids through or across the seal. For example, in some embodiments, the seal or seals can comprise a selective fluid seal that prevents the flow or migration of liquids through or across the seal indefinitely or for a specified period of time while also allowing the flow or migration of gases through or across the seal at the same time. Such selective fluid tight seals (e.g., gas permeable, fluid impermeable seals) can therefore prevent the loss or migration of substrate through the aperture of the base wall when the seal is in a sealing position while also allowing oxygen or other desired gases to pass through the seal into the inner volume of the compartment.

Such selective fluid tight seals can advantageously support incubation, growth, or analyses processes that require the influx of gases into the compartment or substrate. For example, in some embodiments of plant tissue culture, a gas permeable seal can allow proper levels of oxygen and carbon dioxide for cell or plant growth. In some embodiments, a selective fluid tight seal can allow humidity to pass into or out of the compartment in a controlled manner, thus allowing humidity control for a given sample while also providing a fluid tight seal.

In some embodiments, the trays described herein can be used in, or be part of, a system such as an automated system. Automated systems useful with trays described herein include systems that provide automation of filling of the tray with substrate, provide automated addition of biological material to the tray, provide addition of reagents or growth substances or other experimental substances that are to be added to the substrate-filled compartments before or after addition of biological material, provide automated analyses of samples in the tray, provide automated removal or transplant of samples or portions of sample out of the tray or to another location outside of the tray, provide emptying or cleaning of the trays, and the like. In some embodiments, described herein is a system comprising a tray described herein (e.g., such as tray 100), and at least one automated platform selected from an automated substrate dispenser, an automated biological material picker or gripper, an automated analytical platform, an automated sensor, and an automated transplanter. An automated platform can, in some embodiments, include all parts, including hardware and software, necessary for automation of a specific activity, such as, for example, filling the tray with substrate. In some embodiments of the systems described herein, more than one automated platform (including more than one type of automated platform) can be included in the system in order to provide multiple automated processes in one system.

Methods of using the trays described herein are also provided. The various methods for which the trays described herein can be used will become apparent to one skilled in the art based on the disclosure herein. In some embodiments, a method for using the trays described herein can comprise placing biological material in or on a substrate in at least one compartment of a tray described herein (e.g., tray 100). In some embodiments, the method can further comprise a step selected from: storing the biological material in the tray for a period of time, transporting the tray with the biological material from a first location to a second location, allowing the biological material to incubate for a period of time, analyzing at least a portion of the biological material, or combinations thereof.

In some embodiments, a method is provided herein comprising filling at least one compartments (e.g., compartment 110) of a tray described herein (e.g., tray 100) with substrate, such as growth media, and placing biological material in or on the growth media or substrate in at least one compartment of a tray described herein. The method can, in some embodiments, further comprise a step selected from: storing the biological material in the tray for a period of time, transporting the tray with the biological material from a first location to a second location, allowing the biological material to incubate for a period of time, analyzing at least a portion of the biological material, or combinations thereof.

The methods described herein, and various portions or steps of the methods described herein can be completed manually, by an automated process, or by a combination thereof. In some embodiments, one or more of the following steps can be completed manually: filling at least one compartment of the tray, placing biological material in at least one compartment of a tray described herein, removing the seal from at least one compartment of the tray or from a target compartment or from at least a portion of the tray, analyzing at least a portion of the biological material or a portion of the substrate or a portion of a sample (comprising substrate, biological material, and optionally additional substances or reagents), transporting the tray with or without the biological material from a first location to a second location, transferring at least a portion of the biological material, with or without at least a portion of the substrate, to a location outside of the tray, and the like. In some embodiments, one or more of the following steps can be completed using an automated process: filling at least one compartment of the tray, placing biological material in at least one compartment of a tray described herein, removing the seal from at least one compartment of the tray or from a target compartment or from at least a portion of the tray, analyzing at least a portion of the biological material or a portion of the substrate or a portion of a sample (comprising substrate, biological material, and optionally additional substances or reagents), transporting the tray with or without the biological material from a first location to a second location, transferring at least a portion of the biological material, with or without at least a portion of the substrate, to a location outside of the tray, and the like. For example, in some embodiments, an automated substrate dispenser can be used to fill the at least one compartment of the tray.

One exemplary advantage of the trays described herein is their usefulness in analyses, and optionally, transfer, of the biological material, the substrate, or the samples (substrate, biological material, and optionally additional reagents or additives) contained within the compartments. The trays described herein can provide increased options for analyzing the samples contained therein by providing a relatively large aperture in the base wall of each compartment. The aperture also provides options to use tools or machinery, including automated machinery, to elevated or remove the sample for analysis or transfer as described herein. While some tray inserts exist that comprise a bottom aperture, such inserts require additional trays to sit in (and therefore additional material), do not allow segregation of each compartment from each other compartment, and can present difficulties in maintaining the samples securely within their compartments during transport and analysis. The trays described herein solve all of these problems and more. For example, the removable seal provides a low cost, low material solution for holding the substrate in each compartment during filling when the substrate is often in a liquid, liquid-like, or flowable phase. Further, because the seal 130 can be individually seal on the exterior portion 122 of base wall 120, each compartment can be independently filled with substrate (e.g., same or different substrate), samples (e.g., same or different), and reagents (e.g., same or different), without a concern of intermixing between compartments. In the case of plant trays, such segregation between compartments can also prevent root entanglement and parasitic feeding by one plant in one compartment on the nutrients present in another compartment. Additionally, the seal is easy to remove, either manually or in an automated manner, and the base wall provides support to the samples, keeping them in the tray during processing of the trays after removal of the seal or seals.

Methods described herein can comprise a step of analyzing at least a portion of the biological material. Analysis of the samples, substrate, or biological material can take many different forms. For example, various sensors may be used to obtain information from a sample, substrate, or biological material. Sensors can include, for example, cameras, spectrophotometers, multispectral camera, infrared (IR) or near infrared (NIR) cameras, and the like, and combinations thereof. The sensors can be directed to obtain information from a target compartment of a tray, e.g., a compartment from which it is desired that information is obtained about the contents of the compartment, e.g., the sample, the substrate, or the biological material contained therein. In some embodiments of the methods described herein, at least one sensor can be positioned at a location directly above the top open end of a target compartment of the tray. In some embodiments, at least one sensor can be positioned at a location directly below the aperture of a target compartment of the tray. In some embodiments, at least one sensor can be positioned at a location directly above the top open end of a target compartment of the tray and at least one sensor can be positioned at a location directly below the aperture of a target compartment of the tray. Once the at least one sensor is positioned, information about the sample, the substrate, or the biological material can be obtained from the sensor or sensors.

One advantage of the trays described herein is that each compartment is isolated from the other compartments, allowing for wide variety of options for utilizing the compartments, such as in various experimental designs, analyses, and tracking and tracing of specific samples. For example, because of the independent compartments, in some embodiments, the substrate in at least one compartment of the tray can be different from the substrate in at least one other compartment of the tray. The trays described herein can make tracking and tracing of each compartment simpler and allow multiple different samples, substrates, and treatments to be present in the same tray. In some embodiments, different biological samples can be disposed in different compartments on the same tray. For example, at least a first biological sample can be disposed in a first compartment of the tray and at least a second biological sample disposed in a second compartment of the tray, wherein the first and second biological samples are different types of samples, for example, are derived from different organisms, are different plants or plant parts of different varieties, are different species, comprise a different treatment, or are derived from different parts of one or more organisms. In some embodiments, the substrates or the samples can be treated with a treatment, which can be the same or different from compartment to compartment. For example, in some embodiments, a first treatment can be applied to a substrate or biological sample of a first compartment of the tray; and a second treatment can be applied to a substrate or biological sample of a second compartment of the tray, wherein the first and second treatments are different. Exemplary non-limiting treatments can include, nutrition treatments, hormones, chemical reagents, growth agents, pharmaceutical agents, antimicrobial treatments, and the like.

In some embodiments of the methods described herein the seal is removed from the target compartment, before or after at least one analyzing step. In some embodiments, the seal can be removed by, e.g., peeling or pulling the seal off of the exterior portion of the base wall of the compartment, thus exposing the aperture as an open aperture. In some embodiments, this action can be completed manually by a user, or can be completed using an automated mechanism or process. In some embodiments, the seal can be removed by cutting at least a portion of the seal.

In some embodiments, when the seal is cut, the cut portion can be removed from the tray to expose the aperture as an open aperture. Alternatively, in some embodiments, the cut portion can be left in place while the action of cutting the seal effectively removes the sealing capability of the seal. For example, a shape of an outline of the aperture, or of a pushing structure, can be cut into the seal and the cut portion of the seal (e.g., inside of the outline) can be left in place. In such embodiments, because the seal is cut, the aperture can function as an open aperture, and the seal and substrate can, for example, be pushed, together, upwards toward the top open end of the compartment by pushing on the cut portion of the seal and the substrate through the now open aperture. In some such embodiments, the cutting of the seal can be performed by a pushing structure, and the process of removing the seal and pushing on the substrate through the open aperture can be accomplish in a single step or movement. For example, a pushing structure can include a blade at the outline of the pushing structure or in the shape of the aperture outline on top of the pushing structure in order to cut through the seal during a pushing motion. In some embodiments, the pushing structure can be articulated or spun to aid in the cutting of the seal. In some embodiments, the pushing structure can contain a heat-based cutting portion that allows cutting through the seal using heat. In some embodiments, a laser can be used to cut the seal. In some embodiments, the seal can have perforations that allow breaking of the seal upon application of pressure to the seal, such as with a pushing structure.

In some embodiments, removal of the seal allows access for analysis, such as where the seal and the tray are constructed of light-impermeable material, or where the analysis of the sample is best completed by removing or exposing at least a portion of the sample, substrate, or biological material (e.g., a cell or multiple cells, tissue, root tissue, plant tissue, etc.) out of the compartment. For example, in some embodiments of the methods described herein, analyzing comprises at least partially removing a sample comprising at least a portion of the biological material, with or without at least a portion of the substrate, from a target compartment of the tray; positioning at least one sensor in a position that allows collection of information from the sample; and obtaining information about the biological material or the substrate from the at least one sensor. Removal of the sample or biological material, or a portion thereof, from the compartment can allow additional options for analysis beyond the limited options available for in-tray analysis. For example, such removal can allow different or improved views or angles or access to parts of the sample or biological material. In some embodiments, obtaining information about the biological material or the substrate from the at least one sensor can comprise moving the sensor in a 90 degree, 180 degree, or 360 degree rotation around at least one axis of the sample. In such cases, the sensor can obtain multiple points of information around the entire perimeter or circumference of the sample. For example, images can be taken such that a 360 degree image can be obtained, or information about all single-plane sides of the sample can be obtained. In some embodiments, obtaining information about the biological material or the substrate from the at least one sensor can comprise moving the sensor in a 360 degree rotation around two or more axes of the sample. In some embodiments, and entire 3-dimensional image or representation of the sample can be obtained from moving a sensor in a 360 degree rotation around multiple axes of the sample. In some embodiments, the sensor can be a movable sensor. In some embodiments, the sensor can be an automated sensor or a sensor having automated movement capability.

In some embodiments, information obtained from the sensors can include information about the physiochemical status of the biological material. For examples, the information can include information about one or more parameters such as water content, chemical composition or partial chemical composition, phenotype, genotype, temperature, color, size of at least a portion of the biological material, and the like.

Information obtained from sensors can be provided to a user, a user interface, or a system, or the like, via analog or digital methods. In some embodiments, the information can be presented to a user on a graphical user interface. In some embodiments, the obtained information can be stored in a database or transmitted to a device for further processing. In some embodiments of the methods described herein, obtained information can be used to determine, manually or automatically (e.g., by comparing the information against a database, a threshold value, a present value, preexisting images, and the like), what to do with the samples in further steps of the methods. For example, in some embodiments, the information obtained in one or more steps of the processes can be used to determine whether a sample proceeds to another step, is discarded, is ignored, or is transferred to a different location, such as to another tray or receptacle. In some embodiments information obtained during two or more analysis steps of a method described herein can be compiled prior to using the information to make a determination regarding the specific sample or target compartment. It is to be further understood that, in some embodiments, human observation can take the place of sensors in obtaining information about the samples.

In some embodiments of the methods described herein, the sample is removed or at least partially removed from the compartment of the tray in order to perform analysis or to move the sample to another location. The trays described herein advantageously offer multiple ways to access and remove samples, substrate, or biological material contained within a compartment of the tray. For example, removal can be done by gripping the substrate or sample or biological material through the top open end of the compartment and pulling the sample, substrate, or biological materials upward out of the compartment through the top open end. In such methods, a gripper can optionally be used to grip the material (sample, substrate, biological material), and pull at least a portion of it out of the compartment. Thus, in some embodiments of the methods describe herein, removing the sample comprises gripping the biological material or the substrate or a combination thereof through the top open end of the target compartment and pulling at least a portion of the biological material, with or without at least a portion of the substrate, out of the target compartment through the top open end of the target compartment. In some embodiments, the gripping is performed by a manual gripper or a hand. In some embodiments, the gripping is performed by an automated gripper.

Another method of removal can include pushing on the sample or substrate through the aperture in the base wall, thus pushing the sample upward and out through the top open end of the compartment. In some embodiments, pushing on the sample upward can be performed by use of a pushing structure. In some embodiments, removing the sample can comprise removing the seal from a target compartment of the tray to yield an open-aperture target compartment; and inserting a pushing structure into the open-aperture target compartment through the aperture, thereby pushing at least a portion of the substrate and the biological material together through the top open end of the open-aperture target compartment. In some embodiments the pushing structure can have a pushing surface that is approximately the same size and shape as the surface area of the aperture in the base wall of the target compartment. In some embodiments the pushing structure can have a pushing surface that is from about 0.01% to about 10%, from about 0.05% to about 10%, from about 0.1% to about 10%, from about 0.5% to about 10%, from about 1% to about 10%, from about 0.01% to about 5%, from about 0.05% to about 5%, from about 0.1% to about 5%, from about 0.5% to about 5%, or from about 1% to about 5%, smaller than the surface area of the aperture in the base wall of the target compartment. In some embodiments, the pushing structure can be operated manually. In some embodiments, the pushing structure is an automated pushing structure.

In some embodiments, such as where the pushing structure is smaller than the dimension of the surface area of the aperture, a disc or other such piece of low-flexibility material can be set on top of the pushing structure, matching the dimension of, or slightly lesser in dimension than, the surface area of the aperture. This can aid in raising the substrate through the top open end without if falling out around a pushing structure that is smaller in dimension than the aperture, or prevent the pushing structure from becoming lodged in the substrate. In some embodiments, a disc or other such piece of low-flexibility material can be set inside the compartment, resting, for example, on the base wall inside the compartment, prior to filling or partially filling the compartment with substrate. In such cases the disc or other such piece can be greater in dimension than the aperture, allowing sufficient support of various types of substrates, for example, when the pushing structure is pushing on the disc from the bottom. In some embodiments, the substrate can be a loose material, such as soil, and the pushing structure and pushing action can cause at least a portion of the loose substrate material to fall away from the biological material and expose at least a portion of the biological material, or leave a sample comprising at least a portion of the biological material and at least a portion of the substrate that is in total significantly smaller than the original volume of the sum of the substrate and the biological material before at least a portion of the loose substrate material to fell away during pushing up of the substrate by the pushing structure. For example, where the substrate is a loose soil and the biological material is a plant or plant part, the pushing structure can be centered on at least a portion of the plant or plant part such that when the pushing structure pushes up, loose soil can fall away and at least a portion of the plant or plant part can be exposed, or a sample is left comprising the plant or plant part and a portion of the soil such that the sample left is in total significantly smaller than the original volume of the sum of the substrate and the plant or plant part. Such action can, in some embodiments, make activities such as analysis, gripping, and transfer or transplantation of the plant or plant part easier.

In some embodiments, the methods described herein can comprise transferring at least a portion of the biological material, with or without at least a portion of the substrate, to a location outside of the tray or to a same or different location on the same (e.g., original) tray. In some embodiments, the location outside the tray can be selected from a second tray, a receptacle or receiver, an analysis platform, a petri dish, a plate comprising wells, and the like. In some embodiments, the location outside the tray can have a same or different substrate from the original tray. For example, in a plant micropropagation process, a first tray (as described herein) can be used for generation and, optionally, analysis of plantlets in a gel-like growth media, and then the plantlets can be transplanted from the first tray to a second tray or receptacle containing a soil or soil-like substrate. The transfer can, in some embodiments, be performed manually. In other embodiments, the transferring can be done by an automated process, such as by an automated gripper that removes and places the at least a portion of the biological material, with or without at least a portion of the substrate. In some embodiments, the biological material is a seed, a plant, or a plant part, and the transferring comprises transplanting the seed, plant, or plant part into a soil or soil-like substrate. The transplanting can, in some embodiments, be performed manually. In other embodiments, the transplanting can comprise using an automated transplanter, or using an automated placing mechanism, a gripper, an end-effector, a robot or robot arm, a cobot, a carousel, or a combination thereof.

Without wishing to be bound by theory, it is believed that the design of the trays described herein, including the aperture, optional release of the seal, optional slope of the body portion sidewall, and optional texturing on the sidewall can all contribute to different capabilities or ease of removal of the desired sample, whether by gripping and pulling through the top open end, or by pushing through the aperture to push the sample out of the top open end.

The following embodiments are intended to be illustrative of the present disclosure and not limiting.

Embodiment 1 is tray comprising:
a plurality of compartments, each compartment comprising
   a top open end;
   a body portion sidewall; and
   a base wall having at least one aperture, wherein the base wall has an exterior portion, and
a removable fluid tight or selective fluid tight seal disposed on the exterior portion of the base wall of at least one compartment.

Embodiment 2 is the tray of embodiment 1, wherein the aperture is a single aperture, and, optionally, wherein the aperture is disposed in the center of the base wall.

Embodiment 3 is the tray of any one of embodiments 1 or 2, wherein a ratio of a surface area of the base wall measured in the plane of the base wall to a surface area of the aperture measured in the plane of the base wall is from about 1.2:1 to about 5:1, from about 1.2:1 to about 1.5:1, from about 1.2:1 to about 2:1, from about 1.2:1 to about 2.2:1, from about 1.2:1 to about 2.5:1, from about 1.2:1 to about 3:1, from about 1.2:1 to about 3.5:1, from about 1.2:1 to about 4:1, from about 1.2:1 to about 4.5:1, from about 1.5:1 to about 4:1, from about 1.5:1 to about 3.5:1, from about 1.5:1 to about 3:1, from about 1.5:1 to about 2.5:1, from about 1.5:1 to about 2:1, from about 2:1 to about 5:1, from about 2:1 to about 4.5:1, from about 2:1 to about 4:1, from about 2:1 to about 3.5:1, from about 2:1 to about 3:1, or from about 2:1 to about 2.5:1.

Embodiment 4 is the tray of any one of embodiments 1 to 3, wherein the seal is flexible.

Embodiment 5 is the tray of any one of embodiments 1 to 4, wherein the seal comprises a peelable seal.

Embodiment 6 is the tray of any one of embodiments 1 to 5, wherein the seal has a pull tab.

Embodiment 7 is the tray of any one of embodiments 1 to 6, wherein the tray, the lid and/or the seal is optically clear or transparent or opaque, for example, the seal is optically clear or transparent or opaque.

Embodiment 8 is the tray of any one of embodiments 1 to 7, wherein the seal is removably affixed to the exterior portion of the base wall by an adhesive, a heat seal, or a cold seal.

Embodiment 9 is the tray of any one of embodiments 1 to 8, wherein the body portion sidewall slopes inward from the top open end to the base wall.

Embodiment 10 is the tray of embodiment 9, wherein the body portion sidewall slopes inward from the top open end to the base wall with a slope of from about 0.5 degrees to about 5 degrees.

Embodiment 11 is the tray of any one of embodiments 1 to 10, wherein at least a portion of the tray is adapted to engage with a tray lid, e.g. the lid is reversible but firmly connected to the tray.

Embodiment 12 is the tray of any one of embodiments 1 to 11, wherein the tray is a plant tray having a top side and a bottom side.

Embodiment 13 is the tray of embodiment 12, wherein the top side of the tray comprises a lip portion adapted to engage with a tray lid.

Embodiment 14 is the tray of any one of embodiments 1 to 13, wherein the tray is constructed of a material selected from polypropylene, polyethylene, polyvinylchloride, polystyrene, polymethylmethacrylate, polycarbonate, polytetrafluoroethylene, polydimethylsiloxane, polysulfone, or combinations thereof.

Embodiment 15 is the tray of any one of embodiments 1 to 14, wherein the tray and the compartments are constructed as a single piece of material.

Embodiment 16 is the tray of any one of embodiments 1 to 14, wherein the tray further comprises a tray frame, and wherein each compartment is independently constructed of a separate piece of material from the tray frame.

Embodiment 17 is the tray of embodiment 16, wherein each independent compartment is removable.

Embodiment 18 is the tray of any one of embodiments 1 to 17, wherein the tray has from 2 to 384 compartments.

Embodiment 19 is the tray of any one of embodiments 1 to 18, wherein the seal is an independent seal independently disposed on each compartment, such that a tray having a plurality of compartments has a plurality of seals, each independently disposed on each compartment.

Embodiment 20 is the tray of any one of embodiments 1 to 18, wherein the seal is a single seal disposed across 2 or more of the compartments.

Embodiment 21 is the tray of any one of embodiments 1 to 18, wherein the seal is a single seal disposed across the plurality of compartments.

Embodiment 22 is the tray of any one of embodiments 1 to 21, further comprising substrate independently disposed within each compartment.

Embodiment 23 is the tray of embodiment 22, wherein the substrate is selected from a bacterial culture media, a yeast culture media, a plant culture media, and an animal culture media, optionally wherein the substrate comprises one or more gelling agents selected from agar, carrageenan, gellan gum, alginic acid and its salts, agarose, and combinations thereof.

Embodiment 24 is the tray of any one of embodiments 22 or 23, wherein the substrate is a gel or solid at room temperature.

Embodiment 25 is a system comprising:
at least one automated platform selected from an automated substrate dispenser, an automated biological material picker or gripper, an automated analytical platform, an automated sensor, and an automated transplanter; and
a tray according to any one of embodiments 1 to 24 or 59 to 61.

Embodiment 26 is a method for producing a tray, comprising:
applying a removable seal to an exterior portion of a base wall of at least one compartment of a tray comprising:
   a plurality of compartments, each compartment comprising
      a top open end;
      a body portion sidewall; and
      a base wall having at least one aperture; and
sealing the seal to the exterior portion of the base wall of the at least one compartment such that a fluid tight seal is formed over the at least one aperture.

Embodiment 27 is the method of embodiment 26, further comprising filling at least one compartment with substrate.

Embodiment 28 is the method of embodiment 27, wherein the substrate is selected from a bacterial culture media, a yeast culture media, a plant culture media, and an animal culture media, optionally wherein the substrate comprises one or more gelling agents selected from agar, carrageenan, gellan gum, alginic acid and its salts, agarose, and combinations thereof.

Embodiment 29 is the method of any one of embodiments 27 to 28, wherein the substrate is a gel, liquid, semi-solid, or solid at room temperature.

Embodiment 30 is the method of any one of embodiments 27 to 29, wherein the filling of the at least one compartment is performed by an automated filling process.

Embodiment 31 is a method comprising:
placing biological material in or on the substrate in at least one compartment of a tray according to any one of embodiments 22 to 24 or 59 to 61; and
further comprising a step selected from: storing the biological material in the tray for a period of time, transporting the tray with the biological material from a first location to a second location, allowing the biological material to incubate for a period of time, analyzing at least a portion of the biological material, or combinations thereof.

Embodiment 32 is a method comprising:
filling at least one compartment of a tray according to any one of embodiments 1 to 21 with substrate;
placing biological material in or on the substrate in at least one compartment of the tray; and
further comprising a step selected from: storing the biological material in the tray for a period of time, transporting the tray with the biological material from a first location to a second location, allowing the biological material to incubate for a period of time, analyzing at least a portion of the biological material, or combinations thereof.

Embodiment 33 is the method of embodiment 32, wherein an automated substrate dispenser is used to fill the at least one compartment of the tray.

Embodiment 34 is the method of any one of embodiments 31 to 33, wherein the method comprises the step of analyzing at least a portion of the biological material, and wherein analyzing comprises:
positioning at least one sensor at a location directly above the top open end of a target compartment of the tray, or positioning at least one sensor at a location directly below the aperture of a target compartment of the tray; and obtaining information about the biological material or the substrate from the at least one sensor.

Embodiment 35 is the method of embodiment 34, further comprising removing the seal from the target compartment.

Embodiment 36 is the method of any one of embodiments 31 to 33, wherein the method comprises the step of analyzing at least a portion of the biological material, and wherein analyzing comprises:
at least partially removing a sample comprising at least a portion of the biological material, with or without at least a portion of the substrate, from a target compartment of the tray;
positioning at least one sensor in a position that allows collection of information from the sample; and
obtaining information about the biological material or the substrate from the at least one sensor.

Embodiment 37 is the method of embodiment 36, wherein obtaining information about the biological material or the substrate from the at least one sensor comprises moving the sensor in a 360 degree rotation around at least one axis of the sample.

Embodiment 38 is the method of any one of embodiments 36 or 37, wherein removing the sample comprises gripping the biological material or the substrate or a combination thereof through the top open end of the target compartment and pulling at least a portion of the biological material, with or without at least a portion of the substrate, out of the target compartment through the top open end of the target compartment.

Embodiment 39 is the method of embodiment 38, wherein the gripping is performed by an automated gripper.

Embodiment 40 is the method of any one of embodiments 36 to 39, wherein removing the sample comprises:
removing the seal from a target compartment of the tray to yield an open-aperture target compartment; and
inserting a pushing structure into the open-aperture target compartment through the aperture, thereby pushing at least a portion of the substrate and the biological material together through the top open end of the open-aperture target compartment.

Embodiment 41 is the method of any one of embodiments 35 or 40, wherein removing the seal comprises peeling or pulling the seal off of the exterior portion of the base wall of the compartment.

Embodiment 42 is the method of any one of embodiments 35 or 40, wherein removing the seal comprises cutting at least a portion of the seal.

Embodiment 43 is the method of embodiment 42, wherein the cutting at least a portion of the seal is performed with the pushing structure while inserting the pushing structure.

Embodiment 44 is the method of any one of embodiments 40 to 43, wherein the pushing structure has a pushing surface that is approximately the same size and shape as the surface area of the aperture, or from about 0.05% to about 10% smaller than the surface area of the aperture.

Embodiment 45 is the method of any one of embodiments 40 to 44, wherein the pushing structure is an automated pushing structure.

Embodiment 46 is the method of any one of embodiments 34 to 45, wherein the sensor is an automated sensor or a sensor having automated movement capability.

Embodiment 47 is the method of any one of embodiments 34 to 46, wherein the sensor is selected from a camera, a thermometer, a multispectral camera, or combinations thereof.

Embodiment 48 is the method of any one of embodiments 34 to 47, wherein the information includes information about the physiochemical status of the biological material.

Embodiment 49 is the method of any one of embodiments 34 to 48, wherein the information includes information selected from water content, chemical composition or partial chemical composition, phenotype, genotype, temperature, color, or size of at least a portion of the biological material.

Embodiment 50 is the method of any one of embodiments 31 to 49, further comprising transferring at least a portion of the biological material, with or without at least a portion of the substrate, to a location outside of the tray.

Embodiment 51 is the method of embodiment 50, wherein the location outside the tray is selected from a second tray, a receptacle, an analysis platform, a petri dish, or a plate comprising wells.

Embodiment 52 is the method of any one of embodiments 50 or 51, wherein the biological material is a seed, a plant, or a plant part, and wherein transferring comprises transplanting the seed, plant, or plant part into a plant pot, a plug tray, or a plant tray.

Embodiment 53 is the method of any one of embodiments 50 to 52, wherein the transplanting comprises using an automated transplanter.

Embodiment 54 is the method of any one of embodiments 31 to 53, wherein the placing comprises using an automated placing mechanism.

Embodiment 55 is the method of any one of embodiments 31 to 54 wherein the biological material is selected from an eukaryotic cell, a plant cell, an animal cell, a mammalian cell, a prokaryotic cell, a primary cell, a tumor cell, a stem cell, a genetically altered cell, a transformed cell, and an immortalized cell, a tissue or portion of a tissue selected from plant tissue or animal tissue, a plant seed, a protoplast, a cytoplast, a plant organ, or a plant part.

Embodiment 56 is the method of any one of embodiments 31 to 54, wherein the biological material is selected from a seed or a plant part selected from a cutting, an embryo, a regenerated embryo, a colony, a microspore, a callus, or an anther.

Embodiment 57 is the method of any one of embodiments 31 to 56, further comprising applying a first treatment to a substrate or biological sample of a first compartment of the tray; and applying a second treatment to a substrate or biological sample of a second compartment of the tray, wherein the first and second treatments are different.

Embodiment 58 is the method of any one of embodiments 31 to 57, wherein placing biological material in or on the substrate in at least one compartment of the tray comprises placing a first biological sample in or on a substrate of a first compartment of the tray; and placing a second biological sample in or on a substrate of a second compartment of the tray, wherein the first and second biological samples are derived from different organisms, are different plants or plant parts of different varieties, are different species, comprise a different treatment, or are derived from different parts of one or more organisms.

Embodiment 59 is the tray of any one of embodiments 22-24, wherein the substrate in at least one compartment of the tray is different from the substrate in at least one other compartment of the tray.

Embodiment 60 is the tray of any one of embodiments 22-24 or 59, further comprising a biological sample disposed on or in the substrate of at least one compartment of the tray.

Embodiment 61 is the tray of embodiment 60, comprising at least a first biological sample disposed in a first compartment of the tray and at least a second biological sample disposed in a second compartment of the tray, wherein the first and second biological samples are derived from different organisms, are different plants or plant parts of different varieties, are different species, comprise a different treatment, or are derived from different parts of one or more organisms.

Illustrative embodiments of the trays, systems, and methods are discussed herein and reference has been made to some possible variations. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A tray comprising:
a plurality of compartments, each compartment comprising
a top open end;
a body portion sidewall; and
a base wall having one aperture, wherein the base wall has an exterior portion, and
a removable fluid tight or selective fluid tight seal disposed on the exterior portion of the base wall of at least one compartment, and
wherein there is no physical or liquid communication between a sealed compartment and another compartment when the tray is in an upright and level position, and wherein removal of the seal from a particular compartment creates an open-aperture target compartment.

2. The tray of claim 1, wherein a ratio of a surface area of the base wall measured in the plane of the base wall to a surface area of the aperture measured in the plane of the base wall is from about 1.2:1 to about 5:1.

3. The tray of claim 1, wherein the seal is flexible, a peelable seal, or a combination thereof.

4. The tray of claim 1, wherein the seal is removably affixed to the exterior portion of the base wall by an adhesive, a heat seal, or a cold seal.

5. The tray of claim 1, wherein the body portion sidewall slopes inward from the top open end to the base wall, optionally with a slope of from about 0.5 degrees to about 5 degrees.

6. The tray of claim 1, wherein at least a portion of the tray is adapted to engage with a tray lid.

7. The tray of claim 1, wherein the seal is a single seal disposed across 2 or more of the compartments.

8. The tray of claim 1, further comprising substrate independently disposed within each compartment.

9. The tray of claim 8, wherein the substrate is selected from a bacterial culture media, a yeast culture media, a plant culture media, or an animal culture media, optionally wherein the substrate comprises one or more gelling agents selected from agar, carrageenan, gellan gum, alginic acid and its salts, agarose, or combinations thereof.

10. A method comprising:
a step of:
placing biological material in or on a substrate in at least one compartment of a tray according to claim 1, wherein the substrate is independently disposed within each compartment; or
filling at least one compartment of a tray according to claim 1 with a substrate and placing biological material in or on the substrate in at least one compartment of the tray; and
a step selected from: storing the biological material in the tray for a period of time, transporting the tray with the biological material from a first location to a second location, allowing the biological material to incubate for a period of time, analyzing at least a portion of the biological material, or combinations thereof.

11. The method of claim 10, wherein the method comprises the step of analyzing at least a portion of the biological material, and wherein analyzing comprises:
positioning at least one sensor at a location directly above the top open end of a target compartment of the tray, or positioning at least one sensor at a location directly below the aperture of a target compartment of the tray; and
obtaining information about the biological material or the substrate from the at least one sensor,
optionally further comprising removing the seal from the target compartment.

12. The method of claim 10, wherein the method comprises the step of analyzing at least a portion of the biological material, and wherein analyzing comprises:
at least partially removing a sample comprising at least a portion of the biological material, with or without at least a portion of the substrate, from a target compartment of the tray;
positioning at least one sensor in a position that allows collection of information from the sample; and
obtaining information about the biological material or the substrate from the at least one sensor.

13. The method of claim 12, wherein removing the sample comprises:
removing the seal from a target compartment of the tray to yield an open-aperture target compartment; and
inserting a pushing structure into the open-aperture target compartment through the aperture, thereby pushing at least a portion of the substrate and the biological material together through the top open end of the open-aperture target compartment.

14. The method of claim 11, wherein removing the seal comprises cutting at least a portion of the seal, or peeling or pulling the seal off of the exterior portion of the base wall of the compartment.

15. The method of claim 10, further comprising transferring at least a portion of the biological material, with or without at least a portion of the substrate, to a location outside of the tray.

16. The tray of claim 1, wherein at least a portion of the tray is adapted to engage with a tray lid, wherein the tray lid is reversible but firmly connected to the tray.

17. The tray of claim 16, wherein the tray lid is reversible but firmly connected to the tray by a locking mechanism or an hinge.
